(19) United States
(12) Patent Application Publication
(10) Pub. No.: US 2025/0283044 A1
(43) Pub. Date: Sep. 11, 2025

(54) PRODUCTS FOR CELL CONVERSION

(71) Applicant: HIGHERSTEAKS LIMITED, Milton, Cambridgeshire (GB)

(72) Inventors: Benjamina Bollag, Milton, Cambridgeshire (GB); James Clarke, Milton, Cambridgeshire (GB); Ruth Helen Faram, Milton, Cambridgeshire (GB)

(21) Appl. No.: 18/262,403

(22) PCT Filed: Nov. 23, 2021
(86) PCT No.: PCT/EP2021/082719

§ 371 Date: Jul. 21, 2023

(30) Foreign Application Priority Data
Jan. 22, 2021 (GB) 2100879.2
Jan. 22, 2021 (GB) 2100880.0

(51) Int. Cl. C12N 5/077; A23L 13/00; C12N 5/071; C12N 15/113

(52) U.S. Cl. CPC C12N 5/0652; A23L 13/00; C12N 5/0697; C12N 15/113; C12N 2501/65; C12N 2513/00

(57) ABSTRACT

The present invention relates to mRNA molecules and their use in differentiating cells to produce an edible meat product, as well as delivery vehicles for said mRNA molecules.

### CROSS REFERENCE TO RELATED APPLICATIONS

The present application is the U.S. National Phase under 35 U.S.C. § 371 of International Application No. PCT/EP2021/082719, filed Nov. 23, 2021, and entitled “PRODUCTS FOR CELL CONVERSION”, which claims benefit of two United Kindgom Patent Applications, GB2100879.2 and GB2100880.0, both of which were field on Jan. 22, 2021, and entitled “PRODUCTS FOR CELL CONVERSION”. Contents of each of the above documents are incorporated herein by reference for all purposes.

### INCORPORATION OF SEQUENCE LISTING

The present application contains a substitute Sequence Listing submitted in .TXT format via Patent Center and is hereby incorporated by reference in its entirety. Said substitute Sequence Listing was created on Nov. 30, 2023, and is named “Substitute-USSN18262403_SequenceListing.txt”, and is 11 kilobytes in size. Said substitute Sequence Listing in .TXT format is identical to the Sequences disclosed in the PCT parent application, PCT/EP2021/082719. No new matter is introduced.

### FIELD

The present invention relates to mRNA molecules and their use in differentiating cells to produce an edible meat product.

### BACKGROUND

The global population is expected to surpass 9 billion by 2050. Food production may need to substantially increase to fulfill the demand of the growing population, however there are constraints on resources and arable land. Rapidly developing countries such as China, India, and Russia may increase consumption of richer food products, such as meat or other animal products (e.g. dairy, eggs) leading to an increased global demand on these items. According to the report of the Food and Agriculture Organization of the United Nations, the livestock sector is responsible for 18% of Greenhouse Gas (GHG) emissions, uses 30% of earth's terrain, 70% of arable land, and 8% of global freshwater. In addition, the world's demand for meat is expected to double by 2050, rendering traditional meat production systems unsustainable. Compared to several meat sources, particularly beef production, cultured meat may decrease 7-45% of energy use, 78-96% of the GHG emissions, 99% of land use and 82-96% of water use.

Cultured meat products can be produced through in-vitro tissue culture of animal cells in contrast to inefficient traditional livestock agriculture. Multiple cell types may be desirable in creating a cultured meat product, as traditional meat products generally do not solely consist of muscle-derived tissue, but fat, and connective tissue among others. Stem cell differentiation may provide an efficient avenue in producing multiple cell and tissue types for a heterogeneous cultured meat product. Forced, transient gene expression in cells such as stem cells and with simultaneous conditioning and expansion in a bioreactor may result in an efficient and holistic approach in developing a cultured meat product. Provided herein are products, methods and systems for producing edible meat.

### SUMMARY

Provided herein is a non-human animal MYOD1 messenger RNA (mRNA), wherein the MYOD1 mRNA comprises a nucleic acid sequence comprising SEQ ID NO: 1 or a nucleic acid sequence having 70% or more sequence identity to the nucleic acid sequence of SEQ ID NO:1.

In some embodiments, the MYOD1 mRNA is porcine mRNA. In some embodiments, the MYOD1 mRNA comprises a nucleic acid sequence having at least one nucleic acid modification as compared to the nucleic acid sequence of SEQ ID NO: 1.

In some embodiments, the MYOD1 mRNA comprises a nucleic acid sequence having 90% or more sequence identity to the nucleic acid sequence of SEQ ID NO: 1.

In some embodiments, the MYOD1 mRNA comprises at least one nucleic acid modification in the 5′UTR, 3′UTR and/or the length of the poly A-tail.

Also provided herein is a composition comprising the non-human animal MYOD1 mRNA as described anywhere herein. In some embodiments, the composition further comprises siRNA. In some embodiments, the siRNA comprises POU5F1 (OCT3/4), SOX2, nanog, SSEA-4, KLF4 and/or TRA-1-60. In some embodiments, the composition further comprises non-MYOD1 mRNA. In some embodiments, the non-MYOD1 mRNA comprises MRF4, PAX7, PAX3, MYOG, MYF5 and/or MYF6 mRNA. In some embodiments, the composition further comprises combinations of the mentioned siRNA(s) and non-MYOD1 mRNA(s).

Also provided herein is a plasmid comprising the non-human animal MYOD1 mRNA as described anywhere herein. In some embodiments, the plasmid further comprises non-MYOD1 mRNA. In some embodiments, the non-MYOD1 mRNA comprises MYOG1, Mesogenin1, MRF4, PAX7, PAX3, MYOG, MYF5 and/or MYF6 mRNA.

Also provided herein is use of the non-human animal MYOD1 mRNA, composition or plasmid as described anywhere herein, in the production of an edible meat product.

An aspect of the present disclosure provides a method for differentiating non-human stem cells or transdifferentiating non-human somatic cells, to produce an edible meat product, the method comprising: (a) delivering nucleic acid molecules comprising two or more different ribonucleic acid (RNA) molecules into the cells, wherein the RNA molecules comprise non-human animal MYOD1 messenger RNA (mRNA), wherein the MYOD1 mRNA comprises a nucleic acid sequence comprising SEQ ID NO: 1 or a nucleic acid sequence having 70% or more sequence identity to the nucleic acid sequence of SEQ ID NO:1; (b) modulating expression of one or more genes in the cells with aid of the nucleic acid molecules to cause at least a subset of the cells to yield one or more progenitor cells following delivery of the nucleic acid molecules, wherein upon the modulating, the nucleic acid molecules are not integrated into a genome of the cells; (c) culturing the one or more progenitor cells to generate one or more cultured cells; and (d) differentiating or transdifferentiating the one or more cultured cells to generate one or more terminally differentiated cells to produce the edible meat product.

An aspect of the present disclosure provides a method for differentiating non-human stem cells to produce an edible meat product, the method comprising: (a) delivering nucleic acid molecules comprising two or more different ribonucleic acid (RNA) molecules into the cells, wherein the RNA molecules comprise non-human animal MYOD1 messenger RNA (mRNA), wherein the MYOD1 mRNA comprises a nucleic acid sequence comprising SEQ ID NO: 1 or a nucleic acid sequence having 70% or more sequence identity to the nucleic acid sequence of SEQ ID NO:1; (b) modulating expression of one or more genes in the cells with aid of the nucleic acid molecules to cause at least a subset of the cells to yield one or more progenitor cells following delivery of the nucleic acid molecules, wherein upon the modulating, the nucleic acid molecules are not integrated into a genome of the cells; (c) culturing the one or more progenitor cells to generate one or more cultured cells; and (d) differentiating or transdifferentiating the one or more cultured cells to generate one or more terminally differentiated cells to produce the edible meat product.

An aspect of the present disclosure provides a method for transdifferentiating non-human somatic cells to produce an edible meat product, the method comprising: (a) delivering nucleic acid molecules comprising two or more different ribonucleic acid (RNA) molecules into the cells, wherein the RNA molecules comprise non-human animal MYOD1 messenger RNA (mRNA), wherein the MYOD1 mRNA comprises a nucleic acid sequence comprising SEQ ID NO: 1 or a nucleic acid sequence having 70% or more sequence identity to the nucleic acid sequence of SEQ ID NO: 1; (b) modulating expression of one or more genes in the cells with aid of the nucleic acid molecules to cause at least a subset of the cells to yield one or more progenitor cells following delivery of the nucleic acid molecules, wherein upon the modulating, the nucleic acid molecules are not integrated into a genome of the cells; (c) culturing the one or more progenitor cells to generate one or more cultured cells; and (d) differentiating or transdifferentiating the one or more cultured cells to generate one or more terminally differentiated cells to produce the edible meat product.

In some embodiments, the MYOD1 mRNA delivered in the method is porcine mRNA. In some embodiments, the MYOD1 mRNA delivered in the method comprises a nucleic acid sequence having at least one nucleic acid modification as compared to the amino acid sequence of SEQ ID NO: 1. In some embodiments, the MYOD1 mRNA delivered in the method comprises a nucleic acid sequence having 90% or more sequence identity to the nucleic acid sequence of SEQ ID NO: 1. In some embodiments, the MYOD1 mRNA delivered in the method comprises at least one nucleic acid modification in the 5′UTR, 3′UTR and/or the length of the poly A-tail.

In some embodiments, two or more different ribonucleic acid (RNA) molecules are generated via in vitro transcription. In some embodiments, step (d) of the method comprises producing a tissue from the one or more terminally differentiated cells. In some embodiments, the tissue comprises muscle tissue. In some embodiments, the tissue comprises muscle tissue and fat tissue, neural tissue, vascular tissue, epithelial tissue, connective tissue, bone or a combination thereof. In some embodiments, the tissue comprises about from 70 to 90% muscle tissue. In some embodiments, the one or more terminally differentiated cells comprise at least two types of terminally differentiated cells. In some embodiments, the one or more terminally differentiated cells comprise muscle cells. In some embodiments, the method further comprises co-culturing at least two types of terminally differentiated cells to generate a three-dimensional tissue. In some embodiments, at least two types of terminally differentiated cells comprise muscle cells and fat cells, somite cells, neural cells, endothelial cells, smooth muscle cells, bone or cartilage cells, or a combination thereof.

In some embodiments, the RNA molecules further comprise MRF4, PAX7, PAX 3, MYOG, MYF5 or MYF6, or any combination or variant thereof. In some embodiments, the nucleic acid molecules comprise unlocked nucleic acid molecules. In some embodiments, at least one of the RNA molecules is modified with unlocked nucleic acid monomers (uRNAs). In some embodiments, the uRNAs are incorporated at various points along at least one of the RNA molecules. In some embodiments, at least one of the RNA molecules is chemically modified to improve its stability. In some embodiments, chemical modifications to at least one of the RNA molecules comprise anti-reverse cap analogues, 3′-globin UTR, the length of the poly A-tail, or any combination thereof. In some embodiments, the RNA molecules comprise additional non-MYDO1 messenger RNA (mRNA), microRNA (miRNA), transfer RNA (tRNA), silencing RNA (siRNA), self-amplifying RNA (saRNA) or a combination thereof. In some embodiments, the RNA molecules comprise MYOD1 mRNA, and siRNA. In some embodiments, the siRNA comprises POU5F1 (OCT3/4), SOX2, NANOG, SEEA-4, KLF4 and/or TRA-1-60 siRNA. In some embodiments, the RNA molecules comprise MYOD1 mRNA and non-MYOD1 mRNA. In some embodiments, non-MYOD1 mRNA comprises Mrf4, Pax7, PAX3, MYOG, MYF5 and/or MYF6 mRNA. In some embodiments, the RNA molecules comprise MYOD1 mRNA, non-MYOD1 mRNA and siRNA. In some embodiments, the nucleic acid molecules further comprise complementary deoxyribonucleic acid (cDNA) molecules. In some embodiments, the nucleic acid molecules are synthetic nucleic acid molecules. In some embodiments, the nucleic acid molecules are delivered to the stem cells or somatic cells with neutral or anionic liposomes, cationic liposomes, lipid nanoparticles, ionizable lipids, or any combination or variation thereof. In some embodiments, the siRNA comprises POU5F1 (OCT3/4), SOX2, NANOG, SEEA-4, KLF4 and/or TRA-1-60 or any combination or variant thereof.

In some embodiments, steps (c) and (d) of the method are performed in the same bioreactor chamber. In some embodiments, (c) and (d) are performed in different bioreactor chambers. In some embodiments, the method comprises the use of a degradable scaffold. In some embodiments, the degradable scaffold is configured to facilitate cell expansion during the one or more expansion processes in a bioreactor chamber. In some embodiments, step (b) is conducted in the presence of the degradable scaffold. In some embodiments, step (c) is conducted in the presence of the degradable scaffold.

Another aspect of the present disclosure provides a method comprising: (a) modulating expression of one or more genes in the stem cells or somatic cells in a transient and non-integrative manner using two or more ectopic differentiation factors to generate progenitor cells; (b) culturing at least a subset of the progenitor cells to generate cultured cells; and (c) differentiating at least a subset of the cultured cells to generate terminally differentiated cells to produce the edible meat product, wherein said two or more ectopic differentiation factors comprise nucleic acid, wherein the nucleic acid comprises non-human animal MYOD1 messenger RNA (mRNA), wherein the MYOD1 mRNA comprises a nucleic acid sequence comprising SEQ ID NO: 1 or a nucleic acid sequence having 70% or more sequence identity to the nucleic acid sequence of SEQ ID NO:1.

Another aspect of the present disclosure provides a method comprising: (a) modulating expression of one or more genes in the stem cells in a transient and non-integrative manner using two or more ectopic differentiation factors to generate progenitor cells; (b) culturing at least a subset of the progenitor cells to generate cultured cells; and (c) differentiating at least a subset of the cultured cells to generate terminally differentiated cells to produce the edible meat product, wherein said two or more ectopic differentiation factors comprise nucleic acid, wherein the nucleic acid comprises non-human animal MYOD1 messenger RNA (mRNA), wherein the MYOD1 mRNA comprises a nucleic acid sequence comprising SEQ ID NO: 1 or a nucleic acid sequence having 70% or more sequence identity to the nucleic acid sequence of SEQ ID NO:1.

Another aspect of the present disclosure provides a method comprising: (a) modulating expression of one or more genes in the somatic cells in a transient and non-integrative manner using two or more ectopic differentiation factors to generate progenitor cells; (b) culturing at least a subset of the progenitor cells to generate cultured cells; and (c) differentiating at least a subset of the cultured cells to generate terminally differentiated cells to produce the edible meat product, wherein said two or more ectopic differentiation factors comprise nucleic acid, wherein the nucleic acid comprises non-human animal MYOD1 messenger RNA (mRNA), wherein the MYOD1 mRNA comprises a nucleic acid sequence comprising SEQ ID NO: 1 or a nucleic acid sequence having 70% or more sequence identity to the nucleic acid sequence of SEQ ID NO:1.

In some embodiments, (b) and (c) are performed in the same bioreactor chamber. In some embodiments, (b) is performed in a bioreactor chamber and (c) is performed in an additional bioreactor chamber. In some embodiments, the terminally differentiated cells comprise muscle cells, fat cells, somite cells, neural cells, endothelial cells, smooth muscle cells, bone cells, or a combination thereof. In some embodiments, the ectopic differentiation factors comprise nucleic acids, polypeptides, small molecules, growth factors, or any combination thereof. In some embodiments, at least the subset of cultured cells are differentiated by arresting the cell cycle of cells. In some embodiments, the ectopic differentiation factors arrest the cell cycle of cells through reducing or removing growth factors from the subset of cultured cells. In some embodiments, the growth factors comprise LIF, FGF, BMP, activin, MAPK, TGF-β, NRG-1 or any combination or variant thereof. In some embodiments, the arresting the cell cycle of cells occurs by reducing or removing growth factor levels in a solution in which the culturing is conducted.

Provided herein is a method for differentiating non-human stem cells or transdifferentiating non-human somatic cells, to produce an edible meat product, the method comprising:
i) delivering one or more active agents in a colloidal lipid particle composition into said cells;
ii) modulating expression of one or more genes in said cells with said active agent to generate progenitor cells;
iii) culturing at least a subset of said progenitor cells to generate cultured cells; and
iv) differentiating at least a subset of said cultured cells to generate terminally differentiated cells to produce said edible meat product;
wherein said lipid particle composition comprises a cationic lipid comprising from 35-65 mol % of the total lipid present, and one or more structural lipids comprising from 10-30 mol % of the total lipid present.

Also provided herein is a method for differentiating non-human stem cells to produce an edible meat product, the method comprising:
i) delivering one or more active agents in a colloidal lipid particle composition into said cells;
ii) modulating expression of one or more genes in said cells with said active agent to generate progenitor cells;
iii) culturing at least a subset of said progenitor cells to generate cultured cells; and
iv) differentiating at least a subset of said cultured cells to generate terminally differentiated cells to produce said edible meat product;
wherein said lipid particle composition comprises a cationic lipid comprising from 35-65 mol % of the total lipid present, and one or more structural lipids comprising from 10-30 mol % of the total lipid present.

Also provided herein is a method for transdifferentiating non-human somatic cells to produce an edible meat product, the method comprising:
i) delivering one or more active agents in a colloidal lipid particle composition into said cells;
ii) modulating expression of one or more genes in said cells with said active agent to generate progenitor cells;
iii) culturing at least a subset of said progenitor cells to generate cultured cells; and
iv) differentiating at least a subset of said cultured cells to generate terminally differentiated cells to produce said edible meat product;
wherein said lipid particle composition comprises a cationic lipid comprising from 35-65 mol % of the total lipid present, and one or more structural lipids comprising from 10-30 mol % of the total lipid present.

Also provided herein is use of a colloidal lipid particle composition for producing an edible meat product, where said lipid particle comprises
a) one or more active agents
b) a cationic lipid comprising from 35-65 mol % of the total lipid present, and
c) one or more structural lipids comprising from 10-30 mol % of the total lipid present.

Provided herein is a method for differentiating non-human stem cells or transdifferentiating non-human somatic cells, to produce an edible meat product, the method comprising:
i) delivering one or more active agents using a colloidal lipid particle containing composition into said cells;
ii) modulating expression of one or more genes in said cells with said active agent to generate progenitor cells;
iii) culturing at least a subset of said progenitor cells to generate cultured cells; and
iv) differentiating at least a subset of said cultured cells to generate terminally differentiated cells to produce said edible meat product;
wherein said lipid particle-containing composition comprises a cationic lipid comprising from 35-65 mol % of the total lipid present, and one or more structural lipids comprising from 10-30 mol % of the total lipid present.

Also provided herein is a method for differentiating non-human stem cells to produce an edible meat product, the method comprising:
i) delivering one or more active agents using a colloidal lipid particle containing composition into said cells;
ii) modulating expression of one or more genes in said cells with said active agent to generate progenitor cells;
iii) culturing at least a subset of said progenitor cells to generate cultured cells; and
iv) differentiating at least a subset of said cultured cells to generate terminally differentiated cells to produce said edible meat product;
wherein said lipid particle composition comprises at the very least a cationic lipid comprising from 35-65 mol % of the total lipid present, and one or more structural lipids comprising from 10-30 mol % of the total lipid present.

Also provided herein is a method for transdifferentiating non-human somatic cells to produce an edible meat product, the method comprising:
i) delivering one or more active agents in a colloidal lipid particle containing composition into said cells;
ii) modulating expression of one or more genes in said cells with said active agent to generate progenitor cells;
iii) culturing at least a subset of said progenitor cells to generate cultured cells; and
iv) differentiating at least a subset of said cultured cells to generate terminally differentiated cells to produce said edible meat product;
wherein said lipid particle composition comprises at the very least a cationic lipid comprising from 35-65 mol % of the total lipid present, and one or more structural lipids comprising from 10-30 mol % of the total lipid present.

Also provided herein is use of a colloidal lipid particle-containing composition for producing an edible meat product, where said lipid particle comprises:
a) one or more active agents
b) a cationic lipid comprising from 35-65 mol % of the total lipid present, and
c) one or more structural lipids comprising from 10-30 mol % of the total lipid present.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example flow chart schematic wherein an edible biomaterial scaffold and species-specific constructs may be produced, the cells may be expanded in one or a plurality of bioreactors in contact with the scaffolds and constructs, differentiated in one or a plurality of bioreactors, and laminar media flowed and recycled between bioreactor tanks.

FIG. 2A illustrates an exemplary plasmid design, for producing a single RNA molecule encoding four target gene sequences, for encapsulating into a e.g. lipid particle for delivery to a target cell. FIG. 2B illustrates an exemplary production overview of mRNA using said plasmid for FIG. 2A.

FIG. 3 is a schematic to show the relative gene expression profile developments as muscle cells form. Early myogenic cells initially express PAX7. MYOD1, the master muscle regulatory gene, which is responsible for securing the myogenic state, rises soon after and induces the expression of MYOG and MHC (myosin heavy chain). The cells become multinucleated. At the point MHC is expressed, the cells are able to form myofibrils and exhibit cytoskeletal striations.

FIG. 4 shows the results of a gene expression analysis of porcine muscle differentiation.

FIG. 5 shows the results of a protein analysis of porcine muscle differentiation.

FIG. 6 shows the results of a immunohistochemical analysis of porcine muscle cells.

FIG. 7 shows the results of a microscopic analysis of porcine muscle cells.

### DETAILED DESCRIPTION

### 1. Definitions

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The use of the word “a” or “an,” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” is used to indicate that a value includes the inherent variation of error for the device, the method being employed to determine the value, or the variation that exists among the study subjects. Unless otherwise specified based upon the above values, the term “about” means ±5% of the listed value.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “flavour,” as used herein, generally refers to the taste and/or the aroma of a food or drink.

The term “food product,” as used herein, generally refers to a composition that is safe to be ingested by humans or animals, including e.g., domesticated animals (e.g., dogs, cats), farm animals (e.g., cows, pigs, horses), and wild animals (e.g., non-domesticated predatory animals). The term may refer to any substance that can be used or prepared for use as food, such as any substance that can be metabolized by a human or animal to give energy and build tissue. It may be eaten or drunk by any human or animal for nutrition or pleasure. A food product may comprise carbohydrates, fats, proteins, water, or other ingredients which can be safely ingested by humans or animals.

As used herein, the term “nucleic acid” generally refers to a polymeric form of nucleotides of various lengths (e.g., at least 2, 3, 4, 5, 6, 7, 8, 9, 10, 100, 500, 1000 or more nucleotides), either deoxyribonucleotides or ribonucleotides, or analogs thereof. A nucleic acid may include one or more subunits selected from adenosine (A), cytosine (C), guanine (G), thymine (T), and uracil (U), or variants thereof. A nucleotide can include any subunit that can be incorporated into a growing nucleic acid strand. Such subunit can be A, C, G, T, or U, or any other subunit that is specific to one of more complementary A, C, G, T, or U, or complementary to a purine (e.g., A or G, or variant thereof) or pyrimidine (e.g., C, T, or U, or variant thereof). In some examples, a nucleic acid may be single-stranded or double stranded, in some cases, a nucleic acid molecule is circular. Non-limiting examples of nucleic acids include deoxyribonucleic acid (DNA) and ribonucleic acid (RNA). Nucleic acids can include coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), self-amplifying RNA (saRNA), ribozymes, cDNA, recombinant nucleic acids, branched nucleic acids, plasmids, vectors, isolated DNA of any sequence, isolated RNA of any sequence, nucleic acid probes, and primers. A nucleic acid molecule may comprise one or more modified nucleotides, such as methylated nucleotides and nucleotide analogs. A nucleic acid may be synthetic.

The term “cationic lipid” refers to a lipid with a net positive charge at a selected pH, for example a pH of about 7.0.

The term ‘colloidal’ can refer to a mixture in which one substance of microscopically dispersed particles is suspended throughout another substance. The dispersed substance is often referred to as the colloid, with the term colloidal suspension referring to the mixture.

The term ‘bioreactor’ as used herein, refers to a vessel that supports a biologically active environment, for culturing cells on an industrial scale. A bioreactor as used herein is of at least 0.1 m³ capacity, and including up to 500 m³ capacity, i.e. a plant scale bioreactor.

### 2. Overview

The present disclosure provides products and methods for producing tissue engineered food products, such as edible meat products, via the use of a particular mRNA differentiation factor.

The present disclosures also provides methods for producing an edible meat product using colloidal lipid particle compositions. Said particles are used to deliver active agents, such as particular mRNA differentiation factors, to a target cell in vitro, such as a non-human stem cell or non-human somatic cell, wherein the active agents are capable of differentiating said cells to produce an edible meat product.

By using colloidal lipid particles as a vehicle, it is possible to transport active agents across cell membranes. The vehicle increases the stability of the agents and also reduces enzymatic degradation of the active agents as they are protected by the lipid bilayer of the lipid particle. Colloidal lipid particles also do not affect the integrity of cells, since uptake is by innate endocytotic methods and no transient permeabilisation of the membrane occurs. Thus, it is possible to deliver active agents into physiologically stable cells to produce edible meat, without compromising the viability or integrity of the targeted cells. This is particularly important in this field of use, where the cells become the ultimate product and therefore must remain intact.

The present disclosures further provides methods for producing an edible meat product using colloidal lipid particle-containing compositions. Said particles are used to deliver active agents, such as particular nucleic acids to a target cell in vitro, such as a non-human stem cell or non-human somatic cell, wherein the active agents are capable of changing protein expression levels and/or differentiating said cells to produce an edible meat product.

By using colloidal lipid particles as part of a delivery vehicle, it is possible to transport active agents across cell membranes. The vehicle increases the stability of the agents by either encapsulation or chemical interaction, in turn reducing the likelihood of enzymatic degradation of the active agents. Cell integrity is maintained upon uptake by endocytosis given no permeabilisation of the membrane is required. Thus, it is possible to deliver active agents into physiologically stable cells to produce edible meat, without compromising the viability or integrity of the targeted cells. This is particularly important in this field of use, where the mass transfer of cells is paramount to end product

A food product may be any composition that can be ingested and metabolized by humans or animals to give energy and build tissue.

Cultured cells or tissues may be combined with at least one other ingredient. Cultured cells or tissues may be combined with at least one other ingredient to obtain a food product having a desired texture, moisture retention, product adhesion, or any combination thereof. A cultured cell may be a cell grown under controlled conditions such as an in-vitro condition outside their natural environment. An ingredient may comprise a binder, filler, or extender. A filler or binder may comprise a non-meat substance comprising carbohydrates such as a starch. Fillers and binders may include potato starch, flour, eggs, gelatin, carrageenan, and tapioca flour. An extender may have a high protein content. Extenders may comprise soy protein, milk protein, or meat-derived protein. Ingredients that provide flavour, texture, or other culinary properties may be added to a meat product. For example, extracellular matrix proteins may be used to modulate structural consistency and texture. Proteins such as heme or collagen may be incorporated into the extracellular matrix to contribute to the taste and texture of the final food product. Nutrients such as vitamins that are normally lacking in meat products from whole animals may be added to increase the nutritional value of the meat product. This may be achieved either through straight addition of the nutrients to a growth medium or by alternative methods. For example, the enzymes responsible for the biosynthesis of a particular vitamin, such as Vitamin D, A, or the different Vitamin B complexes, may be transfected into the cultured muscle cells to produce the particular vitamin within those cells.

A cultured meat product may be produced by culturing cells in-vitro into a tissue product. A cell may comprise a cell membrane, at least one chromosome, composed of genetic material, cytoplasm, and various organelles which are adapted or specialized to perform one or more vital functions, such as energy and proteins synthesis, respiration, digestion, storage and transportation of nutrients, locomotion, or cell division. A cell may comprise one or a plurality of cells. A cell may comprise a somatic cell, a terminally differentiated cell, a stem cell, a germ cell, or other cell type. A somatic cell may be any cell forming the body of an organism that are not germline cells. Mutations in somatic cells may affect the individual organism but are not passed onto offspring. A cell may comprise satellite cells, myoblasts, myocytes, fibroblasts, hepatocytes, vascular endothelial cells, pericytes, extraembryonic cell lines, somatic cell lines, adipocytes, chondrocytes, somite cells, blood cells, mesenchymal cells, or stem cells. In some embodiments, the somatic cells comprise fibroblasts. In some embodiments, the somatic cells comprise myoblasts. In some embodiments, the somatic cells comprise hematopoietic cells. A myocyte may be the smallest subunit of all muscular tissues. Skeletal muscle myocytes may differentiate from induced pluripotent stem cells and/or mesenchymal stem cells to skeletal muscle myoblasts and fuse into multinucleated muscle fibers, myofibrils, that behave as a unit. These myofibrils may be composed of overlapping filaments, myofilaments, that are both thick and thin and allow for a contraction of its length using a series of motor proteins. An adipocyte may be a cell primarily composed of adipose tissue, specialized in synthesizing and storing energy as fat. Adipocytes may be derived from induced pluripotent stem cells and/or mesenchymal stem cells through adipogenesis. Adipocytes may be white adipocytes, which store energy as a single large lipid droplet and have important endocrine functions, and brown adipocytes which store energy in multiple small lipid droplets but specifically for use as fuel to generate body heat. Cells may be myogenic cells. Myogenic cells may be natively myogenic (e.g. are myogenic cells that are cultured in the cultivation infrastructure). Natively myogenic cells include, but are not limited to, myoblasts, myocytes, satellite cells, side population cells, muscle derived stem cells, mesenchymal stem cells, myogenic pericytes, or mesoangioblasts. Myogenic cells may not be natively myogenic (e.g. are non-myogenic cells that are specified to become myogenic cells in the cultivation infrastructure). Non-myogenic cells include embryonic stem cells, induced pluripotent stem cells, extraembryonic cell lines, and somatic cells other than muscle cells. A cell may be a wild-type cell or may be a genetically modified cell (e.g., transgenic, genome edited). Non-myogenic cells may be modified to become myogenic cells through the expression of one or more myogenic transcription factors such as MYOD1, MYOG, MYF5, MYF6, PAX3, PAX7, paralogs, orthologs, or genetic variants thereof. Myoblast determination protein (MYOD) may be a skeletal muscle specific transcription factor and protein in animals that play a significant role in regulating muscle differentiation. MYOD may commit mesoderm cells to a skeletal myoblast lineage and regulate that differentiation and proliferation of myoblasts. MYOD may be considered a master regulatory gene of skeletal muscle differentiation and its ability to convert fibroblasts and other cell types into skeletal muscle supports its central role in myogenesis.

A cell may comprise a muscle stem cell which may differentiate into specific types of muscle cells such as skeletal muscle cells or smooth muscle cells. Differentiation may refer to the process during which young, unspecialized cells take on individual characteristics and reach their specialized form and function. Cell differentiation may allow a single cell and genotype to result in tens to hundreds of different cell types and phenotypes. Through differentiation, a totipotent cell may move through pluripotency or multipotency, eventually reaching a lineage committed state. A cell may comprise a stem cell which may be any unspecialized cell capable of renewing themselves through cell division which have the developmental potential to differentiate into multiple cell types. A stem cell may be any unspecialized cell capable of self-renewal through cell division which may have the developmental potential to differentiate into multiple cell types, without a specific implied meaning regarding developmental potential, for example a stem cell can be totipotent, pluripotent, multipotent, etc. A stem cell may be a cell capable of proliferation and giving rise to more such stem cells while maintaining its developmental potential. A stem cell may refer to any subset of cells that have the developmental potential, under particular circumstances, to differentiate to a more specialized or differentiated phenotype, and which retain the capacity, under certain circumstances, to proliferate without substantially differentiating. A stem cell may refer to a naturally occurring parent cell whose descendants (progeny cells) specialize, often in different directions, by differentiation, e.g., by acquiring completely individual characters, as occurs in progressive diversification of embryonic cells and tissues. Some differentiated cells may have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype.

A stem cell may be totipotent, pluripotent, multipotent, oligopotent, or unipotent. A stem cell may comprise an embryonic stem cell, animal stem cell, adult stem cell, induced pluripotent stem cell, reprogrammed stem cell, mesenchymal stem cell, hematopoietic stem cell, or a progenitor cell. An embryonic stem cell may refer to embryonic cells capable of differentiating into cells of all three embryonic germ layers (the endoderm, ectoderm and mesoderm), or remaining in an undifferentiated state. The embryonic stem cells may comprise cells which are obtained from the embryonic tissue formed after gestation (e.g., blastocyst) before implantation of the embryo, such as a pre-implantation blastocyst, extended blastocyst cells which are obtained from a post-implantation/pre-gastrulation stage blastocyst, embryonic germ cells which are obtained from the genital tissue of a fetus, and cells originating from an unfertilized ova which are stimulated by parthenogenesis (parthenotes). An embryonic stem cell has unlimited self-renewal ability and pluripotent differentiation ability. An adult stem cell may be any stem cell derived from a somatic tissue of either a post-natal or pre-natal animal. An adult stem cell may be capable of indefinite self-renewal while maintaining its undifferentiated state and is multipotent, capable of differentiation into multiple cell types. Adult stem cells can be derived from any adult, neonatal or fetal tissue such as adipose tissue, skin, kidney, liver, prostate, pancreas, intestine, bone marrow and placenta. Induced pluripotent stem cells or iPSCs may comprise any cells obtained by re-programming of adult somatic cells which are endowed with pluripotency, a cell being capable of differentiating into the three embryonic germ cell layers, the endoderm, ectoderm and mesoderm. Such adult cells may be obtained from any adult somatic tissue (e.g. a skin fibroblast or blood cells) and undergo reprogramming by integrative genetic manipulation or non-integrative protein expression methods, which reset the cell to acquire stem cell-like characteristics. iPSCs may be formed through such processes that reverses the development of the cell or population of cells (e.g., a somatic cell) thus resulting in a naive cell type. An iPSC may be a cell that has undergone a process of driving a cell to a naive state with higher developmental and proliferation potential, such as a cell that is reset into a less differentiated state. The somatic cell, prior to induction to an iPSC, can be either partially or terminally differentiated. There may be a complete or partial reversion of the differentiation state, i.e., an increase in the developmental potential of a cell, to that of a cell having a pluripotent state. A somatic cell may be driven to a pluripotent state, such that the cell has the developmental potential of an embryonic stem cell, similar to an embryonic stem cell phenotype. Induction of a somatic cell may also encompass a partial reversion of the differentiation state or a partial increase of the developmental potential of a cell, such as a somatic cell or a unipotent cell, to a multipotent state. Induction may also encompass partial reversion of the differentiation state of a cell to a state that renders the cell more susceptible to complete induction to a pluripotent state when subjected to additional manipulations. A stem cell may comprise a reprogrammed cell. Cellular reprogramming may be a process that reverses the developmental potential of a cell or population of cells (e.g., a somatic cell). Reprogramming may be a process of driving a cell to a state with higher developmental potential, such as driving a cell backwards to a less differentiated state. The cell to be reprogrammed can be either partially or terminally differentiated prior to reprogramming. Reprogramming may infer a complete or partial reversion of the differentiation state, such as an increase in the developmental potential of a cell, to that of a cell having a pluripotent state, driving a somatic cell to a pluripotent state, such that the cell has the developmental potential of an embryonic stem cell, such as an embryonic stem cell phenotype, or may encompass a partial reversion of the differentiation state or a partial increase of the developmental potential of a cell, such as a somatic cell or a unipotent cell, to a multipotent state. Reprogramming may also encompass a partial reversion of the differentiation state of a cell to a state that renders the cell more susceptible to complete reprogramming to a pluripotent state when subjected to additional manipulations. Hematopoietic stem cells may be adult tissue stem cells, including stem cells obtained from blood or bone marrow tissue of an individual at any age or from cord blood of a newborn individual. These cells may give rise to other blood cells during hematopoiesis. Hematopoietic stem cells may have the ability to self-renew and may be pluripotent, able to generate any and all diverse mature functional hematopoietic cell types such as erythrocytes, platelets, basophils, neutrophils, eosinophils, monocytes, T-lymphocytes, and B-lymphocytes. Mesenchymal stem cells may be multipotent stromal cells that can differentiate into a variety of cell types, including osteoblasts (bone cells), chondrocytes (cartilage cells), myocytes (muscle cells), adipocytes (fat cells which give rise to marrow adipose tissue), and neuron-like cells. Mesenchymal stem cells may be derived from the marrow as well as other non-marrow tissues, such as placenta, umbilical cord blood, adipose tissue, adult muscle, corneal stroma or the dental pulp of deciduous baby teeth. The cells may not have the capacity to reconstitute an entire organ but may be capable of self-renewal while maintaining their multipotency. A progenitor cell may comprise any cell that maintains the ability to differentiate into at least one specific type of cell but is more specific than a stem cell and pushed to differentiate to a target cell. Progenitor cells may not be able to replicate indefinitely and may only divide a limited number of times. A cell may also comprise a reprogrammed cell such as a trans-differentiated mature cell wherein a somatic cell may be reprogrammed or otherwise induced into another lineage without going through an intermediary proliferative stem cell phase. Trans-differentiated mature cells may be somatic cells that are reprogrammed or otherwise induced into another lineage without going through an intermediate proliferative pluripotent stem cell stage. Direct trans-differentiation of mature cells may occur through transient, forced expression of transcription factors, different methods of transfection, culture conditions, and supplementation of small molecules or growth factors.

A cell may be derived from any non-human animals such as mammals (e.g. cattle, buffalo, pigs, sheep, deer, etc.), birds (e.g. chicken, ducks, ostrich, turkey, pheasant, etc.), fish (e.g. swordfish, salmon, tuna, sea bass, trout, catfish, etc.), invertebrates (e.g. lobster, crab, shrimp, clams, oysters, mussels, sea urchin, etc.), reptiles (e.g. snake, alligator, turtle, etc.), or amphibians (e.g. frogs). A cell-derived meat product may comprise one cell type, such as a skeletal muscle myocyte, or a heterogeneous co-culture composition, such as a skeletal muscle myocyte and an adipocyte composition. A plurality of single cell types may be cultured individually and then combined into a final product. A meat product may be derived from muscle cells grown ex vivo and may include fat cells derived also from any non-human animals. A ratio of muscle cells to fat cells may be regulated to produce a meat product with optimal flavor and health effects. A meat product may be derived from myocytes, myoblasts, osteoblasts, osteoclasts, adipocytes, neurons, endothelial cells, smooth muscle cells, cardiomyocytes, fibroblasts, hepatocytes, chondrocytes, kidney cells, cardiomyocytes, or a combination thereof. A meat product may be derived from another cell type. The tissue may comprise a muscle tissue, fat tissue, neural tissue, vascular tissue, epithelial tissue, connective tissue, bone, or a combination thereof. The tissue may comprise another tissue type. A meat product may comprise an organ meat or connective tissue meat such as liver, kidney, heart, tongue, brain, trotters, tripe, sweetmeat, gizzard, caul, sweetbread, pancreas, stomach, lungs, intestine, placenta, chitterlings, testicles, or feet. Regulation may be achieved by controlling the ratio of muscle and fat cells that are initially seeded in culture and/or by varying, as desired, the concentrations and ratio of growth factors or differentiation factors (e.g. mRNA) or other elements that act upon the muscle cells, fat cells, or another cell type.

### 3. Cell Differentiation

The terminally differentiated cells may be used to produce a tissue thus providing the edible meat product. A terminally differentiated cell may be a cell that in the course of acquiring specialized functions, is not able to transform into other types of cells. These cells may constitute a large proportion of the mammalian body and may be unable to proliferate. The terminally differentiated cells may comprise one type of terminally differentiated cells or may comprise at least two types of terminally differentiated cells. In the present invention, the terminally differentiated cells comprise myocyte cells. The two or more types of terminally differentiated cells may comprise myocytes and myoblasts, osteoblasts, osteoclasts, adipocytes, neurons, endothelial cells, smooth muscle cells, cardiomyocytes, fibroblasts, hepatocytes, or chondrocytes. The tissue comprises muscle tissue and optionally fat tissue, neural tissue, vascular tissue, epithelial tissue, connective tissue, bone, or a combination thereof. A muscle tissue may be a form of striated muscle that provides vertebrates with locomotive ability as well as serving metabolic and endocrine roles. Skeletal muscle may be composed of fused and oriented myoblasts which allows a large force to be generated during contraction enabling movement. The skeletal muscle mass of livestock, fish, and game used to produce human food may represent 35-60% of their bodyweight and exhibit a wide diversity in shape, size, anatomical location, and physiological function. Adipose tissue or fat tissue may be a loose connective tissue composed of adipocytes. The main function of adipose tissue may be to store energy in the form of fat. Adipose tissue may be intramuscular or extra muscular. Intramuscular fat content may affect the flavour, juiciness, tenderness, and visual characteristics of meat. There may be a general relationship between the role of increased intramuscular fat and palatability with respects to food products.

A cell phenotype or genotype may be determined using polymerase chain reaction (PCR), immunohistochemistry, or mass spectrometry. The mass spectra obtained from different cells may provide a fine-grained description of the proteomic state of a cell culture or a fingerprint of the cell type which may be used to identify the differentiation states of cells. A determined proteomic fingerprint of cells may be used to characterize other compounds and pinpoint their effect on molecular targets. Mass spectra of cell cultures may require minimal sample preparation, small sample amounts, and provide a high-throughput method of identification for large scale cell cultures enabling rapid identification of cell types. Different desorption and ionization ability in matrix-assisted laser desorption/ionization mass spectrometry (MALDI MS), several pairs of peptides and proteins with similar molecular weight can be regarded as internal standards for each other, especially for those sharing similar structure. The relative intensity of peak pairs detected in the cell lines may be highly conserved. When different species of cells were mixed or co-cultured, the ratiometric peak information can be utilized as a cellular fingerprint for quantitative analysis thus enabling rapid identification and quantification of different cell types according to the ratio values of these peak pairs in mass spectra. Coupled with imaging technology, distribution and proportion of cell types in a whole tissue can be estimated enabling the ratio of different cell types in a heterogeneous tissue in a meat product.

In contrast to traditional livestock agriculture, cells having a self-renewal capacity may be isolated or created and grown in cell culture indefinitely into a tissue structure similar to meat. Such cells may be naturally capable of self-renewal such as embryonic stem cells and pluripotent progenitor cells or may be manipulated to acquire the ability to self-renew. Induced pluripotent stem cells (iPSCs) are stem cells induced from adult somatic cells to obtain a naive pluripotent state, similar to other naive stem cells such as mesenchymal stem cells These cells may be created by reprogramming somatic cells through the introduction of reprogramming factors (transcription factors that drive expression of pluripotency genes). iPSCS are self-replicating and may be passaged and expanded to increase the population. Desired cell types, such as skeletal muscle myocytes or adipocytes, may be generated from iPSCs using manipulation of the cell's environment and differentiation factors. Cultured cells may be directed down a differentiation pathway to generate desired cell types such as into myogenic cells, adipose cells, or other organ cells such as but not limited to hepatocytes, neurons, osteoblasts or osteoblasts. As traditional meat products are not a homogenous composition, rather a heterogeneous combination of multiple tissues and cell types, a population of cells may be differentiated into multiple cell types or independent cell populations may be differentiated into distinct cell types and subsequently combined to produce a composition comprising both muscle and fat cells, or other desired cell types.

Directed differentiation of cells may occur with chemical methods using differentiation factors and small molecules, integrative genetic methods using gene editing techniques to force gene expression, non-integrative (RNA) methods to force gene expression, or integrative or non-integrative viral transduction where viral constructs encoding a gene insert of interest are used to infect and promote forced gene expression. Modulating the expression of one or more genes in a stem cell or somatic cell may comprise the introduction of RNA. “Expression,” “cell expression” or “gene expression” may refer to a process by which information from a gene can be used in the synthesis of a functional gene product. These products may be proteins or may be a functional RNA. Expression may comprise genes transcribed into mRNA and then translated into protein or genes transcribed into RNA but not translated into protein. The RNA introduced may comprise a myogenic gene such as MYOD1, MYOG, MYF5, MYF6, PAX3, PAX7, or any variants, analogs, or combinations thereof. In the invention, the RNA introduced comprises non-human animal MYOD1 mRNA as described anywhere herein. In some embodiments, the two or more RNA target genes are introduced into the cells. In some embodiments, the two or more RNA target genes are introduced via a single RNA molecule.

In some embodiments, the RNA molecule is generated by an in vitro process. In some embodiments, the RNA molecule is produced by in vitro transcription via a single plasmid. In some embodiments, the RNA molecule is produced by in vitro transcription via a single plasmid to incorporate two or more mRNA gene targets into a single RNA molecule.

Generating a single RNA molecule comprising multiple gene targets via a single plasmid improves protein expression potential, reduces transfection number, and ensures consistent protein expression over multiple cell divisions. The plasmid acts as a template for in vitro transcription. In vitro transcription produces the desired single stranded mRNA molecule that can then be delivered into cells.

The plasmid may further comprise key non-structural proteins required for viral self-replication, the sequences of which are taken from non-infectious self-replicating viral RNA. This creates a self-replicating mRNA molecule, with multiple protein expression. The plasmid may further comprise a promotor region, and Internal Ribosome Entry Sites required for CAP-independent translation, allowing multiple genes to be transcribed simultaneously. The plasmid may further comprise a 3′ UTR sequence and a poly-A tail.

### Delivery

RNA may be introduced or delivered into a cell using an expression vector. A vector may comprise any nucleic acid molecule capable of transporting another nucleic acid to which it has been linked.

A vector may comprise a plasmid, which may be a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors may include cosmids, bacterial artificial chromosomes (BAC) and yeast artificial chromosomes (YAC). A vector may comprise a viral vector, wherein additional DNA segments may be ligated into the viral genome. Some vectors may be capable of autonomous replication in a host cell into which they are introduced (e.g., vectors having an origin of replication which functions in the host cell). Other vectors can be integrated into the genome of a host cell upon introduction into the host cell and are thereby replicated along with the host genome. Some vectors may be capable of directing the expression of genes to which they are operatively linked. Expression may be stable or transient. Stable or transient expression may be achieved through stable or transient transfection, lipofection, lipid nanoparticles, electroporation or infection with recombinant viral vectors. Transfection may be the introduction of a heterologous nucleic acid into eukaryote cells, both higher and lower eukaryote cells, as well as yeast and fungal cells. Transfection deliberately introduces nucleic acids into eukaryotic cells artificially to enable the expression or production of proteins using the cell's own machinery or to down-regulate the production of a specific protein by stopping translation.

### Lipid Particles

In some embodiments, the vector is a colloidal lipid particle, wherein the RNA is encapsulated into a lipid particle or self-assembled with a lipid composition. In some embodiments, the colloidal lipid particle is a lipid nanoparticle. In some embodiments, the lipid particle is a liposome. In some embodiments, the colloidal lipid particle is a transferosome. In some embodiments, the colloidal lipid particle is a niosome. In some embodiments, the colloidal lipid particle is an ethosome. In some embodiments, the colloidal lipid particle is a microsphere. In some embodiments, the colloidal lipid particle is a micelle. In some embodiments, the colloidal lipid particle is a dendimer.

In some embodiments, the colloidal lipid particle comprises non-human animal MYOD1 mRNA as described anywhere herein. In some embodiments, the colloidal lipid particle comprises one or more active agents as described anywhere herein. In some embodiments, the colloidal lipid particle comprises non-human animal MYOD1 mRNA as described anywhere herein and one or more additional active agents as described anywhere herein.

In some embodiments, the colloidal lipid particle comprises:
a) the non-human animal mRNA as described anywhere herein
b) a cationic lipid comprising from 35-65 mol % of the total lipid present, and
c) one or more structural lipids comprising from 10-30 mol % of the total lipid present.

In some embodiments, the lipid composition comprises non-lipid components.

### Cationic Lipids

The lipid particles disclosed herein comprise a cationic lipid.

In some embodiments, the cationic lipid is selected from one or more of the following: 1,2-dilinoleyloxy-N,N-dimethylaminopropane (DLinDMA), 1,2-dilinolenyloxy-N,N-dimethylaminopropane (DLenDMA), dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylamino buty 1)-[1,3]-dioxo lane (DLin-K-C4-DMA), 2,2-dilinoleyl-5-dimethylaminomethyl-[1,3]-dioxane (DLin-K6-DMA), 2,2-dilinoleyl-4-Nmethylpepiazino-[1,3]-dioxolane (DLin-K-MPZ), 2,2-dili-noleyl-4-dimethylaminomethyl-[1,3]-dioxolane (DLin-KDMA), 1,2-dilinoleylcarbamoyloxy-3-dimethylaminopropane (DLin-C-AP), 1,2-dilinoleyoxy-3-(dimethylamino acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoley lthio-3-dimethylaminopropane (D Lin-S-D MA), 1-linoleoyl-2-linoleyloxy-3-dimethylaminopropane (DLin-2-D MAP), 1,2-dilinoley loxy-3-trimethy laminopropane chloride salt (DLin-TMA.Cl), 1,2-dilinoleoyl-3-trimethy-laminopropane chloride salt (DLin-TAP.Cl), 1,2-dilinoleyloxy-3-(N-methylpiperazino) propane (DLin-MPZ), 3-(N,Ndilinoleylamino)-1,2-propanediol (DLinAP), 3-(N,Ndioleylamino)-1,2-propanedio (DOAP), 1,2-dilinoleyloxo-3-(2-N,N-dimethylamino) ethoxypropane (DLin-EG-DMA), N,N-dioleyl-N,N-dimethylammonium chloride (DO DAC), 1,2-dioleyloxy-N,N-dimethylaminopropane (DODMA), 1,2-disteary loxy-N,N-dimethy laminopropane (DSD MA), N-(1-(2,3-dioley loxy) propy 1)-N,N,N-trimethy ammonium chloride (DOTMA), N,N-distearyl-N,N-dimethylammonium bromide (DDAB), N-(1-(2,3-dioleoyloxy) propyl)-N,N,N-trimethylanunonium chloride (DOTAP), 3-(N-N′,N′dimethylaminoethane)-carbamoyl) cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido) ethy 1]-N,N-dimethy 1-1-propanaminiumtrifiuoroacetate (DOSPA). Dioctadecylamidoglycyl spermine (DOGS), 3-dimethylamino-2-(cholest-5-en-3-beta-oxybutan-4-oxy)-1-(cis,cis-9, 12-octadecadienoxy)propane (CLinDMA), 2-[5′-(cholest-5-en-3-beta-oxy)-3′-oxapentoxy)-3-dimethyl-1-(cis,cis-9′1-2′-octadecadienoxy) propane (CpLinDMA), N,N-dimethyl-3,4-dioleyloxybenzylamine (DMOBA), 1,2-N,N′dioleylcarbamyl-3-dimethylaminopropane (DOcarbDAP), 1,2-N,N′-dilinoleylcarbamyl-3-dimethy laminopropane (DLincarbDAP), or mixtures thereof.

In some embodiments, the cationic lipid is selected from one or more of DOTAP (DOTAP methosulfate, N-(2,3-Dioleoyloxy-1-propyl) trimethylammonium methyl sulfate), DDA (Dimethyldioctadecylammonium (Bromide Salt)), DLin-KC2-DMA (KC2)(2-[2,2-bis[(9Z,12Z)-octadeca-9,12-dienyl]-1,3-dioxolan-4-yl]-N,N-dimethylethanamine), DLin-MC3-DMA (MC3)((6Z,9Z,28Z,31Z)-heptatriacont-6,9,28,31-tetraene-19-yl 4-(dimethylamino) butanoate), cKK-E12(3,6-bis(4-(bis(2-hydroxydodecyl)amino)butyl)piperazine-2,5-dione) and/or C12-200(.1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol).

The cationic lipid comprises from about 35 mol % to about 65 mol % of the total lipid present in the particle. In some embodiments, the cationic lipid comprises from about 50 mol % to about 65 mol %, from about 50 mol % to about 60 mol %, from about 40 mol % to about 65 mol %, from about 40 mol % to about 60 mol %, from about 40 mol % to about 55 mol %, or from about 40 mol % to about 50 mol % of the total lipid present in the particle. In some embodiments, the cationic lipid comprises from about 35 mol % to about 60 mol %, from about 35 mol % to about 55 mol %, from about 35 mol % to about 50 mol %, or from about 35 mol % to about 45 mol % of the total lipid present in the particle.

In other embodiments, the cationic lipid may comprise from about 35 mol % to about 65 mol % of the total lipid present in the particle.

In some embodiments, the cationic lipid is DOTAP or DOTMA, and comprises from about 35 mol % to about 65 mol % of the total lipid present. In some embodiments, the cationic lipid is DOTAP, and comprises from about 40 mol % to 50 mol % of the total lipid present. In some embodiments, the cationic lipid is DOTMA, and comprises from about 40 mol % to 50 mol % of the total lipid present.

### Structural Lipids

The lipid particles disclosed herein comprise one or more structural lipids. Structural lipids support the formation and stability of particles during and after manufacture.

In some embodiments, the one or more structural lipids are selected from one or more of the following: DOPE (1,2-Distearoyl-sn-glycero-3-phosphocholine), DSPC (1,2-distearoyl-sn-glycero-3-phosphorylcholine), SQDG (glycolipid), MGDG (monogalactosyldiacylglycerol)), DGDG (digalactosyldiacylglycerol), Cyclo-PC (1-palmitoyl-2-cis-9,10-methylenehexadecanoyl-sn-glycero-3-phosphocholine) and/or DGTS (Diacylglyceryltrimethylhomo-Ser).

The structural lipids comprise from about 10 mol % to about 50 mol % of the total lipid present in the particle. In some embodiments, the structural lipids may comprise from about 10 mol % to about 40 mol %, from about 10 mol % to about 30 mol %, from about 15 mol % to about 40 mol %, from about 15 mol % to about 30 mol %, or from about 15 mol % to about 25 mol % of the total lipid present in the particle.

In other embodiments, the one or more structural lipids may comprise from about 10 mol % to about 50 mol % of the total lipid present in the particle.

In some embodiments, the one or more structural lipids is DSPC, and comprise from about 10 mol % to about 50 mol % of the total lipid present. In some embodiments, the one or more structural lipids is DSPC, at about 10 mol % of the total lipid present.

### Other Features

In some embodiments, the lipid particle further comprises a PEG-lipid. The PEG-lipid is useful in that it prevents the aggregation of particles.

PEG is a linear, water-soluble polymer of ethylene PEG repeating units with two terminal hydroxyl groups and are classified by their molecular weights. In some embodiments, the PEG moiety of the PEG-lipid comprises an average molecular weight of from about 600 to about 5,000 Daltons. In some embodiments the average molecular weight is 2,000 Daltons. In some embodiments the average molecular weight is 750 Daltons.

In some embodiments, the PEG-lipid is selected from one or more of the following: PEG-Maleimide, PEG-PDP, PEG-Biotin, PEG-Amine, PEG-DBCO, PEG-Azide, PEG-Cyanur, PEG-Succinyl, PEG-Folate and/or PEG-Carboxylic acid.

In some embodiments, the PEG-lipid is 1,2-DMG PEG 2000 (1,2 dimyristoyl-sn-glycero-3-phosphoethanolamine-N-[methoxy-(polyethylene glycol)-2000.

In some embodiments, the PEG-lipid is a combination of 1,2-DMG PEG2000 and 1,3-DMG PEG2000 (1,3-DMG PEG2000 (1,3 dimyristoyl-sn-glycero-3-phosphoethanolamine-N-[methoxy-(polyethylene glycol)-2000), optionally in a 97:3 ratio.

In some embodiments, the PEG-lipid is 1,2-DMG PEG 1000 (1,2 dimyristoyl-sn-glycero-3-phosphoethanolamine-N-[methoxy-(polyethylene glycol)-1000].

In some embodiments, the PEG-lipid is 1,2-DMG PEG 3000 (1,2 dimyristoyl-sn-glycero-3-phosphoethanolamine-N-[methoxy-(polyethylene glycol)-3000].

In some embodiments, the PEG-lipid comprises from about 1 mol % to about 10 mol % of the total lipid present in the particle. In some embodiments, the sterol may comprise from about 1 mol % to about 5 mol %, 1 mol % to about 3 mol %, about 1 mol % to about 2 mol %, about 1 mol % to 1.5 mol % of the total lipid present in the particle. In some embodiments, the PEG-lipid comprises 2 mol % of the total lipid present. In some embodiments, the PEG-lipid comprises 1.5 mol % of the total lipid present.

In other embodiments, the PEG-lipid comprises from about 1 mol % to about 5 mol % of the total lipid present in the particle.

In some embodiments, the PEG-lipid is 1,2-DMG PEG 2000 (1,2 dimyristoyl-sn-glycero-3-phosphoethanolamine-N-[methoxy-(polyethylene glycol)-2000 and comprises from about 1 mol % to about 2 mol % of the total lipid present.

In some embodiments, the PEG-lipid is a combination of 1,2-DMG PEG2000 and 1,3-DMG PEG2000 (1,3-DMG PEG2000 (1,3 dimyristoyl-sn-glycero-3-phosphoethanolamine-N-[methoxy-(polyethylene glycol)-2000), and comprises from about 1 mol % to about 2 mol % of the total lipid present.

In some embodiments, the PEG-lipid is 1,2-DMG PEG 1000 (1,2 dimyristoyl-sn-glycero-3-phosphoethanolamine-N-[methoxy-(polyethylene glycol)-1000], and comprises from about 1 mol % to about 2 mol % of the total lipid present.

In some embodiments, the PEG-lipid is 1,2-DMG PEG 3000 (1,2 dimyristoyl-sn-glycero-3-phosphoethanolamine-N-[methoxy-(polyethylene glycol)-3000], and comprises from about 1 mol % to about 2 mol % of the total lipid present.

In some embodiments, the lipid particle further comprises a sterol. In some embodiments, the sterol is a cholesterol or a derivative thereof.

Examples of cholesterol derivatives include, but are not limited to, cholestanol, cholestanone, cholestenone, coprostanol, cholesteryl-2′-hydroxyethyl ether, cholesteryl-4′-hy-droxybutyl ether, and mixtures thereof. The synthesis of cholesteryl-2′-hydroxyethyl ether is described herein.

In some embodiments, the sterol comprises from about 10 mol % to about 50 mol % of the total lipid present in the particle. In some embodiments, the sterol may comprise from about 10 mol % to about 20 mol %, about 10 mol % to about 15 mol %, about 30 mol % to about 40 mol % of the total lipid present in the particle. In some embodiments, the sterol comprises about 38 mol % of the total lipid present.

In other embodiments, the sterol may comprise from about 30 mol % to about 40 mol % of the total lipid present in the particle.

In some embodiments, the sterol is cholesterol or a derivative thereof and comprises from about 30 mol % to about 40 mol % of the total lipid present.

In some embodiments, the lipid particle further comprises one or more stabilising agents. Stabilizing agents ensure integrity of the lipid mixture. In some embodiments, the one or more stabilizing agents are polyethylene glycol-lipids. Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA).

The colloidal lipid particles used herein are substantially less-toxic to mammals.

In some embodiment, the lipid particles have a mean diameter of from about 40 nm to about 150 nm. In some embodiments, the diameter is from about 50 nm to about 140 nm, about 60 nm to about 130 nm, or to about 60 nm to about 120 nm.

In the lipid particles of the invention, the active agents are encapsulated within the lipid portion of the lipid particles, thereby protecting the agents from degradation, e.g. enzymatic degradation. In some embodiments, the active agents are fully encapsulated within the lipid particle.

In some embodiments, the lipid:active agent ratio (mass/mass ratio) is from about 1 to about 100. In some embodiments the ratio is from about 1 to about 50 or from about 5 to about 10.

In some embodiments, the lipid components of the colloidal lipid particle depends on the overall charge of the active agent. The charge and size of the active agent directly influences the rate and success of encapsulation, therefore the overall composition and chosen ratios of each lipid component be changed depending on the active molecule in question.

The colloidal lipids particles as described anywhere herein may be prepared by any method known in the art, including, but not limited to, a continuous mixing method or a direct dilution process.

In some embodiments, a plurality of colloidal lipid particles as described anywhere herein are provided for the use of the invention. In some embodiments, said plurality of particles are homogenous in size distribution.

Colloidal lipid particles of the disclosure can combine an active agent as described anywhere herein; a cationic lipid as described anywhere herein; and one or more structural lipids as described anywhere herein.

In some embodiments, the cationic lipid is DOTAP and comprises 40 mol % to 50 mol % of the total lipid present, the structural lipid is DSPC and comprises 10 mol of the total lipid present, and the lipid particle further comprises cholesterol at 30 mol % to 40 mol % of the total lipid present, and one or more PEG-lipids at 1 mol to 2 mol % of the total lipid present.

In some embodiments, the cationic lipid is DOTMA and comprises 40 mol % to 50 mol % of the total lipid present, the structural lipid is DSPC and comprises 10 mol % of the total lipid present, and the lipid particle further comprises cholesterol at 30 mol % to 40 mol % of the total lipid present, and one or more PEG-lipids at 1 mol % to 2 mol % of the total lipid present.

### Viral Constructs

Introduction of nucleic acids by viral infection may have higher transfection efficiencies than other methods such as lipofection and electroporation. Transfection with viral or non-viral constructs may comprise using adenovirus, lentivirus, Herpes simplex I virus, Sendai Virus, or adeno-associated virus (AAV) and lipid-based systems. A lipid may be one or more molecules (e.g., biomolecules) that include a fatty acyl group (e.g., saturated or unsaturated acyl chains). A lipid may include oils, phospholipids, free fatty acids, phospholipids, monoglycerides, diglycerides, and triglycerides. Useful lipids for lipid-mediated transfer of the gene may comprise, DOTMA, DOPE, and DC-Choi. Nucleotides may be delivered by neutral or anionic liposomes, cationic liposomes, lipid nanoparticles, ionizable lipids, or any combinations or variations thereof. A preferred construct may comprise viral vectors such as adenoviruses, AAV, lentiviruses, or retroviruses. A viral construct such as a retroviral construct may include at least one transcriptional promoter/enhancer or locus defining element(s), or other elements that control gene expression by other approaches, such as alternate splicing, nuclear RNA export, or post-translational modification of messenger. A vector construct may further comprise a packaging signal, long terminal repeats (LTRs) or portions thereof, or positive and negative strand primer binding sites appropriate to the virus used. A construct may also include a signal sequence for secretion of the peptide from a host cell in which it is placed. A signal sequence may comprise a mammalian signal. Other non-viral vectors can be used such as cationic lipids, polylysine, or dendrimers. An expression construct may comprise the necessary elements for the transcription and translation of an inserted coding sequence. An expression construct may further comprise sequences engineered to enhance stability, production, purification, or yield of the expressed peptide. For example, the expression of a fusion protein or a cleavable fusion protein comprising the MYODl and/or myogenin (MYOG) protein of some and a heterologous protein can be engineered. Prokaryotic or eukaryotic cells can be used as host-expression systems to express polypeptides of interest such as microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus (CaMV); tobacco mosaic virus (TMV)) or transformed with recombinant plasmid expression vectors, such as Ti plasmid, containing the coding sequence. Mammalian expression systems can also be used to express polypeptides of interest.

### Differentiation Factors

Forced, transient, non-integrative gene expression can be achieved using various nucleic acid molecules such as messenger ribonucleic acid (mRNA), complementary deoxyribonucleic acid (cDNA), micro RNA (miRNA), transfer RNA (tRNA), silencing RNA (siRNA), self-amplifying RNA (saRNA) or any variants, combinations, or analogs thereof. A nucleic acid may be natural in origin or may be a synthetic nucleic acid molecule. Gene expression may be transient, non-integrative such that nucleic acid molecules delivered into a cell are not integrated into the genome of the cell.

mRNA introduced into a cell may make a protein by translation which may be sufficient to differentiate a naïve stem cell into a mature cell type or transdifferentiate a somatic cell. mRNA can be used to differentiate a cell such as with an induced pluripotent stem cell (iPSC) to a skeletal muscle myocyte or transdifferentiate a mature cell such as a fibroblast to a skeletal muscle myocyte. In particular, mRNA can be used to differentiate iPSCs into early myogenic cells and then further into myotubes and myofibrils, where the cells exhibit cytoskeletal striations and multinucleation, to form muscle cells. This process may be called the muscle regulatory cascade. The gene expression profile of the cells also changes during this differentiation process. Myogenic cells initially express high levels of PAX7, followed by MYOD1. As the cells differentiate into myotubes and myofibrils, expression levels of PAX7 and MYOD1 decrease and levels of MYOG and MHC increase. mRNA differentiation protocols may be short (e.g., up to 14 days) and may not cause or harbor adverse effects since mRNAs are otherwise degraded and do not integrate with the host cell genome. mRNA may be a single stranded RNA molecule that corresponds to the genetic sequence of a gene and may be read by the ribosome in the process of transcription. mRNA may be complementary to one of the DNA strands of a gene. An mRNA molecule may carry a portion of the DNA code to other parts of the cell for processing. Physiological mRNA iscreated during transcription wherein a single strand of DNA is decoded by RNA polymerase, synthesizing mRNA. Synthetic mRNA may be created during in vitro transcription (IVT) wherein a single strand of DNA is decoded by RNA polymerase, synthesizing mRNA.

In some embodiments, the mRNA is non-human animal MYOD1 mRNA, wherein the MYOD1 mRNA comprises a nucleic acid sequence comprising the sequence set forth in SEQ ID NO: 1.

SEQ ID NO: 1
ATGGAGCTGCTGAGCCCCCCCCTGAGGGACGTGGACCTGACCGGCCCCGACGGCAGCC
TGTGCAACTTCGCCACCGCCGACGACTTCTACGACGACCCCTGCTTCGACAGCCCCGAC
CTGAGGTTCTTCGAGGACCTGGACCCCAGGCTGGTGCACGTGGGCGCCCTGCTGAAGCC
CGAGGAGCACAGCCACTTCCCCGCCGCCGCCCACCCCGCCCCCGGCGCCAGGGAGGAC
GAGCACGTGAGGGCCCCCAGCGGCCACCACCAGGCCGGCAGGTGCCTGCTGTGGGCCT
GCAAGGCCTGCAAGAGGAAGACCACCAACGCCGACAGGAGGAAGGCCGCCACCATGA
GGGAGAGGAGGAGGCTGAGCAAGGTGAACGAGGCCTTCGAGACCCTGAAGAGGTGCA
CCAGCAGCAACCCCAACCAGAGGCTGCCCAAGGTGGAGATCCTGAGGAACGCCATCAG
GTACATCGAGGGCCTGCAGGCCCTGCTGAGGGACCAGGACGCCGCCCCCCCCGGCGCC
GCCGCCGCCTTCTACGCCCCCGGCCCCCTGCCCCCCGGCAGGGGCGGCGAGCACTACAG
CGGCGACAGCGACGCCAGCAGCCCCAGGAGCAACTGCAGCGACGGCATGATGGACTAC
AGCGGCCCCCCCAGCGGCGCCAGGAGGAGGAACTGCTACGACGGCACCTACTACAGCG
AGGCCCCCAGCGAGCCCAGGCCCGGCAAGAACGCCGCCGTGAGCAGCCTGGACTGCCT
GAGCAGCATCGTGGAGAGCATCAGCACCGAGAGCCCCGCCGCCCCCGCCCTGCTGCTG
GCCGACACCCCCAGGGAGAGCAGCCCCGGCCCCCAGGAGGCCGCCGCCGGCAGCGAGG
TGGAGAGGGGCACCCCCACCCCCAGCCCCGACGCCGCCCCCCAGTGCCCCGCCAGCGC
CAACCCCAACCCCATCTACCAGGTGCTGTGA

The MYOD1 mRNA of the invention has increased translation activation, stability, reduced immunogenicity and decreased degradation.

In some embodiments, the MYOD1 mRNA comprises a nucleic acid sequence having at least 70% sequence identity across the whole sequence compared to the nucleic acid sequence of SEQ ID NO: 1. In some embodiments, the sequence identity is at least 70%, 80%, 90%, 95%, 98%, 99% or 100% across the whole sequence compared to the nucleic acid sequence of SEQ ID NO: 1. In some embodiments, the sequence identity is at least 95% across the whole sequence compared to the nucleic acid sequence of SEQ ID NO: 1.

In some embodiments, the MYOD1 mRNA comprises a nucleic acid sequence comprising at least one nucleic acid modification (e.g., by substitution, addition, or deletion) compared to the nucleic acid sequence of SEQ ID NO: 1. In some embodiments, the nucleic acid sequence comprises at least 1, at least 2, at least 3, at least 4, at least 5, at least 6, at least 7, at least 8, at least 9, or at least 10 nucleic acid modifications compared to the nucleic acid sequence of SEQ ID NO: 1. In some embodiments, the nucleic acid sequence comprises at least one modification in the coding region of the mRNA. In some embodiments, the nucleic acid sequence comprises at least one modification in the 5′ and/or 3′UTR of mRNA.

In some embodiments, the nucleic acid sequence comprises a poly A tail modification to the 3′ end of the mRNA. In some embodiments, the poly A tail is between 25 to 250 nucleic acids in length. The poly A tail modification increases the stability of the mRNA and prevents its degradation in the cell.

In some embodiments, the at least one modification has no impact on the ultimate amino acid sequence that is translated from the mRNA. In some embodiments, the MYOD1 mRNA is codon optimized. Codon optimization may be carried out by any well-known methods in the art. The aim of codon optimization is to increase stability, reduce degradation and promote translation of the mRNA.

In some embodiments, the MYOD1 mRNA is non-human mammalian MYOD1 mRNA (e.g. cattle, buffalo, pigs, sheep, deer, etc.), bird MYOD1 mRNA (e.g. chicken, ducks, ostrich, turkey, pheasant, etc.), fish MYOD1 mRNA (e.g. swordfish, salmon, tuna, sea bass, trout, catfish, etc.), invertebrate MYOD1 mRNA (e.g. lobster, crab, shrimp, clams, oysters, mussels, sea urchin, etc.), reptile MYOD1 mRNA (e.g. snake, alligator, turtle, etc.), or amphibian MYOD1 mRNA (e.g. frogs). In some embodiments the MYOD1 mRNA is porcine MYOD1 mRNA.

In some embodiments, MYOD1 mRNA initiates the muscle regulatory cascade when administered to stem cells. In some embodiments, MYOD1 mRNA initiates expression of MYOD1 and/or MYOG and/or MYH2 in differentiating stem cells. In some embodiments, MYOD1 mRNA initiates myosin heavy chain protein production when administered to stem cells.

siRNA may be a class of short, double stranded RNA non-coding RNA molecules which may interfere with the expression of specific genes with complementary nucleotide sequences. siRNA may interfere with gene expression by degrading mRNA after transcription, preventing translation. siRNAs may be 20-24 base pairs in length with phosphorylated 5′ ends and hydroxylated 3′ ends. siRNAs may target complementary mRNA for degradation, thus preventing translation.

Micro RNA (miRNA) can be small non-coding RNA molecules that function in RNA silencing and post-transcriptional regulation of gene expression. miRNAs base-pair with complementary sequences within mRNA molecules, silencing the mRNA molecules. Silencing may be achieved upon binding of the miRNA to the 3′UTR of the target mRNA through cleavage of the mRNA strand into two pieces, destabilization of mRNA through shortening the poly-A tail, or through inefficient translation of the mRNA into proteins by ribosomes. Modulation of myogenic gene expression may occur through miRNAs. miRNAs that may modulate myogenic gene expression may comprise miR-1, miR-24, miR-26a, miR-27b, miR-29b/c, miR-125b, miR-133, miR-181, miR-206, miR-208b/499, miR-214, miR-221/222, miR-322/424, mi486, or miR-503. These miRNAs may be specifically expressed in cardiac and skeletal muscles under the control of the myogenic transcription factors SRF, MyoD or MEF2 where they may regulate processes of skeletal myogenesis such as myoblast/satellite cell proliferation and differentiation.

Transfer RNA (tRNAs) are adaptor molecules important to translation composed of RNA which serve as a physical link between an mRNA and an amino acid sequence of proteins by carrying an amino acid to the ribosome as directed by a 3-nucleotide codon in a mRNA. tRNAs may be essential for the initiation of protein synthesis by catalyzing ligation of each amino acid to its cognate tRNAs. The translational functions of these entities may be necessary for myogenesis and myogenic differentiation/proliferation. tRNAs that may modulate myogenic gene expression may comprise leucyl-tRNA synthetase, the tRNA gene for lysine, or the tRNA gene for phenylalanine.

cDNA may be a DNA copy synthesized from a single-stranded RNA molecule such as mRNA or miRNA, and produced by reverse transcriptase, a DNA polymer that can use either DNA or RNA as a template. A cDNA can be delivered (e.g., transfected) into a cell to transfer the cDNA that codes for a protein of interest to the recipient cell. A nucleic acid molecule may be delivered to a cell or stem cell to modulate expression of one or more genes in the cells. The modulation may be in a transient and non-integrative manner such that the nucleic acid molecules are not integrated into a genome of the cells. Progenitor cells may be generated following delivery of the cDNA molecules.

Forced human MYOD1 expression may sometimes differentiate human iPSCs and fibroblasts to skeletal muscle myocytes in 7 days with certain constructs. However, protocols used for human cells may not be directly transferable to non-human species. Additionally, novel mRNA transcripts may need to be produced to improve and guarantee species-specific expression using distinct gene sequences for individual species based on various mRNA expression structures such as in cis-acting elements from 5′ to 3′, cap structure, 5′UTR, coding regions with modified nucleotides, 3′ UTR, and a poly-A tails. Species accuracy may improve overall efficiency of the expression system. For example, a porcine viral vector and mRNA sequence in porcine cell culture may provide a more efficient expression system than a human viral vector and mRNA sequence in a porcine cell culture.

In some aspects, the present disclosure provides a method for differentiating stem cells or transdifferentiating somatic cells to produce an edible meat product, the method comprising delivering nucleic acid molecules comprising two or more different ribonucleic acid (RNA) molecules or a single RNA molecule comprising two or more target genes, into the cells, wherein said RNA molecule(s) comprise MYOD1 mRNA as described anywhere herein; modulating expression of one or more genes in the cells with aid of the nucleic acid molecules to cause at least a subset of the cells to yield one or more progenitor cells following delivery of the nucleic acid molecules, wherein upon modulating, the nucleic acid molecules are not integrated into a genome of the cells; culturing the one or more progenitor cells to generate one or more cultured cells; and differentiating the one or more cultured cells to generate one or more terminally differentiated cells to produce the edible meat product.

Differentiating the one or more cultured cells to generate one or more terminally differentiated cells to produce the edible meat product may comprise producing a tissue from the one or more terminally differentiated cells. Cell culturing and differentiating may be performed in a same bioreactor chamber. A bioreactor may be any manufactured device or system which supports a biologically active environment. A bioreactor may be a container suitable for the cultivation of eukaryotic cells, such as mammalian animal cells, or tissues in the context of cell culture. A bioreactor may culture various cell types together, in parallel, or may culture only one cell type singularly. A bioreactor may comprise one vessel or a plurality of vessels and may recycle media used during culture. Culturing at least a subset of progenitor cells or all progenitor cells to generate cultured cells and differentiating at least a subset of the cultured cells to generate terminally differentiated cells to produce an edible meat product may be performed in the same bioreactor chamber or differentiating at least a subset of the cultured cells to generate terminally differentiated cells to produce an edible meat product may be performed in an additional bioreactor.

Modulating expression of one or more genes in the stem cells or the somatic cells may comprise using two or more different messenger RNAs (mRNAs) to generate progenitor cells. For example, forced expression of both PAX7 and MYOD1 together may result in a higher percentage of overall skeletal muscle cells in culture. Modulating expression of one or more genes in the stem cells or the somatic cells may comprise using two or more different messenger RNAs to generate progenitor cells using PAX7 or MYOD1. Furthermore, suppression of pluripotent genes with silencing RNAs (siRNA) can enhance skeletal muscle formation from iPSCs. The transient modulation of expression of one or more genes in a stem cell or a somatic cell may comprise RNA modifications using siRNA(s). An siRNA may comprise POU5F1 (OCT3/4), SOX2, NANOG, SEEA-4, KLF4, TRA-1-60 or any variant, combinations, or analogues thereof. For example, a siRNA targeting OCT3/4 (POU5F1), a pluripotent master regulator, may increase the efficiency of MYOD1 mRNA forced expression.

Delivery of two or more (and particularly three) mRNAs that encode multiple muscle regulatory proteins, results in a more stable and higher protein expression, resulting in a quicker and more reliable differentiation technique.

In some embodiments, the RNA molecules comprise the MYOD1 mRNA, as described anywhere herein, and siRNA. In some embodiments, the siRNA comprises POU5F1 (OCT3/4) siRNA.

In some embodiments, the RNA molecules comprise the MYOD1 mRNA as described anywhere herein, and non-MYOD1 mRNA. In some embodiments, the non-MYOD1 mRNA comprises MYOG. In some embodiments, the non-MYOD1 mRNA comprises Mesogenin 1. In some embodiments, the non-MYOD1 mRNA comprises MYF5. In some embodiments, the non-MYOD1 mRNA comprises MYF4. In some embodiments, the non-MYOD1 mRNA comprises MYOG1. In some embodiments, the non-MYOD1 mRNA comprises MYF6. In some embodiments, the non-MYOD1 mRNA comprises PAX7 mRNA. In some embodiments, the non-MYOD1 mRNA comprises MYF5 mRNA and MYF6 mRNA. In some embodiments, the non-MYOD1 mRNA comprises MYF5 mRNA, MYF6 and PAX7 mRNA. In some embodiments, the RNA molecules comprise the MYOD1 mRNA as described anywhere herein, siRNA and non-MYOD1 mRNA. In some embodiments, the siRNA and non-MYOD1 mRNA may be selected from any one or combination of Mesogenin1, Mesp1, Mesp2, MRF4, MYOG1, SOX2 and PAX3.

As described above, in some embodiments, RNA molecules encoding two or more RNA target genes may be produced via a single plasmid and in vitro transcription. In some embodiments, said RNA molecules comprise the MYOD1 mRNA as described anywhere herein, and any one of, and up to four of, the following porcine mRNA sequences:

Myogenin (MYOG) (SEQ ID NO: 2):
ATGGAGCTGTATGAGACATCCCCCTACTTCTACCAGGAACCCCACTTCTATGACGGGGA
AAACTACCTGCCCGTCCACCTCCAGGGCTTTGAGCCACCAGGCTACGAGCGGACTGAGC
TGAGTCTGAGCCCTGAGGCCCGAGTGCCCCTGGAAGATAAGGGGCTGGGGACCCCCGA
GCACTGCCCAGGCCAGTGCCTGCCGTGGGCATGTAAGGTGTGTAAGAGGAAGTCCGTGT
CTGTGGACCGTCGGCGGGCCGCCACGCTGAGGGAGAAGCGCAGGCTCAAGAAGGTGAA
TGAGGCCTTTGAGGCCCTGAAGAGGAGCACCCTGCTCAACCCCAACCAGCGGCTGCCCA
AGGTGGAGATCCTGCGCAGCGCCATCCAGTACATCGAGTGCCTGCAGGCCCTGCTCAGC
TCCCTCAACCAGGAGGAGCGAGACCTCCGCTACCGAGGCGGGGGGGGCCGCAGCCAG
GGGTGCCCAGTGAATGCAGTTCCCACAGCGCCTCCTGCAGTCCAGAATGGGGCAGTGCA
CTGGAGTTCGGCCCCAACCCAGGGGATCATCTGCTCACAGCTGACCCTACAGATGCCCA
CAATCTGCACTCCCTCACCTCCATCGTGGACAGCATCACAGTGGAGGATGTGGCTGTGG
CCTTCCCAGATGAAACCATGCCCAACTGA
Mesogenin 1 (SEQ ID NO: 3):
ATGGACAACCTGAGGGAGACCTTCCTGAGCCTGGAGGACGGCCTGGGCAGCAGCGACA
GCCCCGGCCTGCTGAGCAGCTGGGACTGGAAGGACAGGGCCGGCCCCTTCGAGCTGAA
CCAGGCCAGCCCCAGCCAGAGCCTGAGCCCCGCCCCCAGCCTGGAGAGCTACAGCAGC
AGCCCCTGCCCCGCCGTGGCCGGCCTGCCCTGCGAGCACGGCGGCGCCAGCAGCGGCG
GCAGCGAGGGCTGCAGCGTGGGCGGCGCCAGCGGCCTGGTGGAGGTGGACTACAACAT
GCTGGCCTTCCAGCCCACCCACCTGCAGGGCGGCGGCGGCCCCAAGGCCCAGAAGGGC
ACCAAGGTGAGGATGAGCGTGCAGAGGAGGAGGAAGGCCAGCGAGAGGGAGAAGCTG
AGGATGAGGACCCTGGCCGACGCCCTGCACACCCTGAGGAACTACCTGCCCCCCGTGTA
CAGCCAGAGGGGCCAGCCCCTGACCAAGATCCAGACCCTGAAGTACACCATCAAGTAC
ATCGGCGAGCTGACCGACCTGCTGAACAGGGGCAGGGAGCCCAGGGCCCAGAGCGCC
MYF5 (SEQ ID NO: 4):
ATGGACGTGATGGACGGCTGCCAGTTCAGCCCCAGCGAGTACTICTACGACGGCAGCTG
CATCCCCAGCCCCGAGGGCGAGTTCGGCGACGAGTTCGTGCCCAGGGTGGCCGCCTTCG
GCGCCCACAAGGCCGAGCTGCAGGGCAGCGACGAGGACGAGCACGTGAGGGCCCCCAC
CGGCCACCACCAGGCCGGCCACTGCCTGATGTGGGCCTGCAAGGCCTGCAAGAGGAAG
AGCACCACCATGGACAGGAGGAAGGCCGCCACCATGAGGGAGAGGAGGAGGCTGAAG
AAGGTGAACCAGGCCTTCGAGACCCTGAAGAGGTGCACCACCACCAACCCCAACCAGA
GGCTGCCCAAGGTGGAGATCCTGAGGAACGCCATCAGGTACATCGAGAGCCTGCAGGA
GCTGCTGAGGGAGCAGGTGGAGAACTACTACAGCCTGCCCGGCCAGAGCTGCAGCGAG
CCCACCAGCCCCACCAGCAACTGCAGCGACGGCATGCCCGAGTGCAACAGCCCCGTGT
GGAGCAGGAAGAGCAGCACCTTCGACAGCATCTACTGCCCCGACGTGAGCAACGTGTA
CGCCACCGACAAGAACAGCCTGAGCAGCCTGGACTGCCTGAGCAACATCGTGGACAGG
ATCACCAGCAGCGAGCAGCCCGGCCTGCCCCTGCAGGACCTGGCCAGCCTGAGCCCCGT
GGCCAGCACCGACAGCCAGCCCGCCACCCCCGGCGCCAGCAGCAGCAGGCTGATCTAC
CACGTGCTG
MRF4 (SEQ ID NO: 5):
ATGATGATGGACCTGTTCGAGACCGGCAGCTACTTCTTCTACCTGGACGGCGAGAACGT
GACCCTGCAGCCCCTGGAGGTGGCCGAGGGCAGCCCCCTGTACCCCGGCAGCGACGGC
ACCCTGAGCCCCTGCCAGGACCAGATGCCCCCCGAGGCCGGCAGCGACAGCAGCGGCG
AGGAGCACGTGCTGGCCCCCCCCGGCCTGCAGCCCCCCCACTGCCCCGGCCAGTGCCTG
ATCTGGGCCTGCAAGACCTGCAAGAGGAAGAGCGCCCCCACCGACAGGAGGAAGGCCG
CCACCCTGAGGGAGAGGAGGAGGCTGAAGAAGATCAACGAGGCCTTCGAGGCCCTGAA
GAGGAGGACCGTGGCCAACCCCAACCAGAGGCTGCCCAAGGTGGAGATCCTGAGGAGC
GCCATCAGCTACATCGAGAGGCTGCAGGACCTGCTGCACAGGCTGGACCAGCAGGAGA
AGATGCAGGAGCTGGGCGTGGACCCCTTCAGCTACAGGCCCAAGCAGGAGAACCTGGA
GGGCGCCGACTTCCTGAGGACCTGCAGCAGCCAGTGGCCCAGCGTGAGCGACCACAGC
AGGGGCCTGGTGATCACCGCCAAGGAGGGCGGCGCCAGCATCGACAGCAGCGCCAGCA
GCAGCCTGAGGTGCCTGAGCAGCATCGTGGACAGCATCAGCAGCGAGGAGAGGAAGCT
GCCCTGCGTGGAGGAGGTGGTGGAGAAG
MYOG1 (SEQ ID NO: 6):
ATGGAGCTGTACGAGACCAGCCCCTACTTCTACCAGGAGCCCAGGTTCTACGACGGCGA
GAACTACCTGCCCGTGCACCTGCAGGGCTTCGAGCCCCCCGGCTACGAGAGGACCGAG
CTGACCCTGAGCCCCGAGGCCCCCGGCCCCCTGGAGGACAAGGGCCTGGGCACCCCCG
AGCACTGCCCCGGCCAGTGCCTGCCCTGGGCCTGCAAGGTGTGCAAGAGGAAGAGCGT
GAGCGTGGACAGGAGGAGGGCCGCCACCCTGAGGGAGAAGAGGAGGCTGAAGAAGGT
GAACGAGGCCTTCGAGGCCCTGAAGAGGAGCACCCTGCTGAACCCCAACCAGAGGCTG
CCCAAGGTGGAGATCCTGAGGAGCGCCATCCAGTACATCGAGAGGCTGCAGGCCCTGC
TGAGCAGCCTGAACCAGGAGGAGAGGGACCTGAGGTACAGGGGCGGCGGCGGCCCCC
AGCCCGGCGTGCCCAGCGAGTGCAGCAGCCACAGCGCCAGCTGCAGCCCCGAGTGGGG
CAGCGCCCTGGAGTTCAGCGCCAACCCCGGCGACCACCTGCTGACCGCCGACCCCACCG
ACGCCCACAACCTGCACAGCCTGACCAGCATCGTGGACAGCATCACCGTGGAGGACGT
GAGCGTGGCCTTCCCCGACGAGACCATGCCCAAC
PAX7 (SEQ ID NO: 7):
ATGGCCGCCCTGCCCGGCACCGTGCCCAGGATGATGAGGCCCGCCCCCGGCCAGAACT
ACCCCAGGACCGGCTTCCCCCTGGAGGTGAGCACCCCCCTGGGCCAGGGCAGGGTGAA
CCAGCTGGGCGGCGTGTTCATCAACGGCAGGCCCCTGCCCAACCACATCAGGCACAAG
ATCGTGGAGATGGCCCACCACGGCATCAGGCCCTGCGTGATCAGCAGGCAGCTGAGGG
TGAGCCACGGCTGCGTGAGCAAGATCCTGTGCAGGTACCAGGAGACCGGCAGCATCAG
GCCCGGCGCCATCGGCGGCAGCAAGCCCAGGCAGGTGGCCACCCCCGACGTGGAGAAG
AAGATCGAGGAGTACAAGAGGGAGAACCCCGGCATGTTCAGCTGGGAGATCAGGGACA
GGCTGCTGAAGGACGGCCACTGCGACAGGAGCACCGTGCCCAGCGTGAGCAGCATCAG
CAGGGTGCTGAGGATCAAGTTCGGCAAGAAGGAGGAGGACGACGAGGCCGACAAGAA
GGAGGACGACAGCGAGAAGAAGGCCAAGCACAGCATCGACGGCATCCTGGGCGACAA
GGGCAACAGGCTGGACGAGGGCAGCGACGTGGAGAGCGAGCCCGACCTGCCCCTGAAG
AGGAAGCAGAGGAGGAGCAGGACCACCTTCACCGCCGAGCAGCTGGAGGAGCTGGAG
AAGGCCTTCGAGAGGACCCACTACCCCGACATCTACACCAGGGAGGAGCTGGCCCAGA
GGACCAAGCTGACCGAGGCCAGGGTGCAGGTGTGGTTCAGCAACAGGAGGGCCAGGTG
GAGGAAGCAGGCCGGCGCCAACCAGCTGGCCGCCTTCAACCACCTGCTGCCCGGCGGC
TTCCCCCCCACCGGCATGCCCACCCTGCCCCCCTACCAGCTGCCCGACAGCACCTACCC
CACCACCACCATCAGCCAGGACGGCGGCAGCACCGTGCACAGGCCCCAGCCCCTGCCC
CCCAGCACCATGCACCAGGGCGGCCTGGCCGCCGCCGCCGCCGCCGCCGACACCAGCA
GCGCCTACGGCGCCAGGCACAGCTTCAGCAGCTACAGCGACAGCTTCATGAACCCCGCC
GCCCCCAGCAACCACATGAACCCCGTGAGCAACGGCCTGAGCCCCCAGGTGATGAGCA
TCCTGAGCAACCCCAGCGCCGTGCCCCCCCAGCCCCAGGCCGACTTCAGCATCAGCCCC
CTGCACGGCGGCCTGGACAGCGCCACCAGCATCAGCGCCAGCTGCAGCCAGAGGGCCG
ACAGCATCAAGCCCGGCGACAGCCTGCCCACCAGCCAGAGCTACTGCCCCCCCACCTAC
AGCACCACCGGCTACAGCGTGGACCCCGTGGCCGGCTACCAGTACGGCCAGTACGGCC
AGACCGCCGTGGACTACCTGGCCAAGAACGTGAGCCTGAGCACCCAGAGGAGGATGAA
GCTGGGCGAGCACAGCGCCGTGCTGGGCCTGCTGCCCGTGGAGACCGGCCAGGCCTAC
MYF6 (SEQ ID NO: 8):
ACTAATTAAATGCCATCTGGGTGGCTCCTCTGGGTTTTTGAGCCCATCACCCAGTTCAGA
CCGAGTCAGAGGCCAAGGAGGAGAACATGATGATGGACCTTTTTGAAACTGGCTCCTAT
TTCTTCTATTTGGACGGGGAAAATGTTACCCTGCAGCCCCTAGAAGTGGCAGAAGGCTC
TCCTTTGTATCCAGGGAGTGATGGTACCCTGTCCCCCTGCCAGGACCAAATGCCCCCGG
AAGCTGGGAGCGACAGCAGTGGAGAGGAACATGTCCTGGCGCCCCCAGGCCTGCAGCC
TCCCCACTGCCCCGGCCAATGTCTGATCTGGGCTTGCAAGACCTGCAAGAGAAAATCTG
CCCCAACCGACCGCAGGAAGGCGGCCACTCTGCGCGAGAGGAGGCGGCTGAAGAAAAT
CAACGAGGCCTTCGAGGCACTGAAGCGGCGGACTGTGGCCAACCCCAACCAAAGGCTG
CCCAAGGTGGAGATCCTGCGGAGCGCCATCAACTACATCGAGAGGTTGCAGGACCTGC
TGCACCGGCTGGATCAGCAGGACAAAATGCAGGAGCTAGGCGTGGACCCCTTCAGCTA
CAGACCCAAGCAAGAGAATCTTGAGGGTGCGGATTTCCTGCGCACCTGCAGCTCCCAGT
GGCCAAGTGTTTCGGATCATTCCAGGGGGCTCGTGATGACTGCCAAGGAAGGAGGGAC
AAACATTGATTCGTCAGCCTCGAGCAGCCTTCGGTGCCTTTCTTCCATCGTGGACAGCAT
TTCCTCGGAGGAACACACGCTCCCCTGCGTCGAGGAAGTGGGGGAGAAATAACTCGCG
GCCGGAGACGGTCTCCACGCAGCAGCAAAAGCCCACCCTCCTCCTCCTCCTCCTCCTCC
GCCTAATCCTGTAGATGAGGTCACGTTACGTGAATATTTAGGAACCCTGACTCAGGAGC
TCACGAAAGGGAAGGGGACATCTTCGCAAAGAAACTTCTCGGAAGCTGTTGCGCACGC
TCGGAGGAGAAGCCTCGCAGCCTTGGGCTTTTCTTCGGCGAACTGCGAGTGGCTTAGAT
CTACAGCAGCCTTGGTTTTTGCTGGGTGGGCTCTGTAACATATTTACGTTTCCTATGGTG
ATCCTTTTGTGCCCTGTGCAAAAGAAGTTCATTCCTGTCTAAAGCAAAGTGGGAACGTC
GCAACTGTTAGTGGGATTGAATGTATTTTTGTAAATAATCTTAGTACTTTCATTTTTTTAT
GTCAACCTAAGAAATATATTTTAAACGTGGAGTGACGTATTGTATACATAGCGTGCAAG
GATCCTGGTATTGTTATATTAAAAAGATAAGTTTCTATA

A nucleic acid molecule may comprise an unlocked nucleic acid molecule. An RNA molecule may be modified. A modification to a nucleic acid, such as an RNA molecule, may comprise modification with unlocked nucleic acid monomers (uRNAs). An individual or a plurality of nucleic acids may be modified with a uRNA. A uRNA may be a small RNA molecule found within the splicing speckles and Cajal bodies of the cell nucleus in eukaryotic cells. uRNAs are generally short, around 150 nucleotides in length, and function in processing pre-messenger RNA in the nucleus. uRNAs are abundant and non-coding. uRNAs may remove introns from pre-mRNAs through successive phosphoryl transfer reactions and make up a spliceosome complex, generating a diversity of mRNA isoforms from each coding gene. A uRNA is a ribonucleoside homologue that lacks a C2′-C4′ bond found in ribonucleosides and is therefore flexible. A uRNA may not lock the ribose moiety in the C3′-endo conformation and incorporation of uRNAs into duplexes may be destabilizing. uRNA monomers may be useful in tuning the specificity and potency of siRNAs without affecting cell viability.

The nucleic acid molecules may comprise unlocked nucleic acid molecules. At least one of the nucleic acid molecules may be modified with unlocked nucleic acid monomers. A uRNA may be incorporated at various points along at least one of the nucleic acid molecules, such as at least one of the RNA molecules.

RNA may be chemically modified for example to improve its stability. Eukaryotic mRNA may comprise a coding region flanked by a 5′ and 3′ untranslated regions (UTRs), as well as a 5′ 7-methylguanosine triphosphate cap and a 3′ poly-A tail which may be necessary in mRNA stability and translation. A chemical modification to improve RNA stability may comprise anti-reverse cap analogues, 3′-globin UTR, or poly-A tail length. A capped or anti-reverse capped mRNA may have enhanced translational efficiency. Cap analogues may comprise modifications to the 5′ end of an mRNA by addition of 7-Methylguanosine (N7-methyl guanosine (m7G). Cap analogues may be incorporated in reverse orientation with the methylated G proximal to the RNA which may result in an inability to translate mRNA transcripts. An anti-reverse capped analogue may not be incorporated in reverse orientation as they contain only one 3′-OH group rather than the two 3′-OH groups in the initial cap analogues and may increase translational efficiency over a conventional cap analogue. An anti-reverse capped analogue may comprise a 3′-O-methyl, 3′-H, or 2′-O-methyl modification in the 7-methylguanosine, or N2 modifications (benzyl or 4-methoxybenzyl). Eukaryotic mRNA transcripts include 5′ and 3′ untranslated regions (UTRs) which may comprise regulatory elements. RNA stability and translational efficiency may be improved by incorporating 5′ and 3′ UTRs. A UTR may comprise alpha-globin or beta-globin mRNAs. Beta-globin 5′ and 3′ UTRs may improve translational efficiency and alpha-globin 3′ UTRs may stabilize mRNA. A poly-A tail may be added to the 3′ end of eukaryotic mRNA transcripts during transcription which may regulate mRNA stability and translation synergistically with the m7G cap by binding poly-A binding protein forming a complex with the m7G cap. A poly-A tail may be encoded on the DNA template from which the mRNA is transcribed, or recombinant poly-A polymerase may be used to extend the mRNA after transcription. Increasing the length of the poly-A tail may increase the efficiency of polysome formation as well as the level of protein expression.

In some aspects, the present disclosure provides a method for generating an edible meat product using stem cells or somatic cells. The method may comprise delivering into the stem cells or somatic cells two or more different types of nucleic acid molecules, wherein said nucleic acid molecules comprise the MYOD1 messenger RNA (mRNA) as described anywhere herein. Non-limiting examples of further nucleic acid molecules which may be delivered into the cells comprise, e.g., non-MYOD1 messenger ribonucleic acid (mRNA), microRNA (miRNA), transfer RNA (tRNA), silencing RNA (siRNA), self-amplifying RNA (saRNA), complementary deoxyribonucleic acid (cDNA), or any combination or variant thereof. The nucleic acid molecules can be delivered into the cells. The nucleic acid may degrade in the cell. The nucleic acid molecules may not pose any significant adverse effects to the cells. Following delivery of the nucleic acid molecules, expression of one or more genes in the cells may be altered or modulated (e.g., with the aid of or due to the presence of the nucleic acid molecules). The alteration or modulation may comprise enhancing, reducing, or inhibiting the gene expression. The alteration or modulation may be in a transient or non-integrative manner such that the nucleic acid molecules are not integrated into a genome of the cells. Such alteration or modulation of gene expression may cause at least a subset of the cells to yield one or more progenitor cells. Some or all of the progenitor cells may subsequently be cultured to generate cultured cells, which cultured cells may be differentiated to generate terminally differentiated cells. The terminally differentiated cells can be used to produce an edible meat product.

The two or more different types of nucleic acid molecules may be generated by an in vitro process, for example using a single plasmid comprising two or more nucleic acid sequences. The two or more different types of nucleic acid molecules comprise the MYOD1 mRNA as described anywhere herein and may comprise further non-MYOD1 mRNA and/or siRNA. The non-MYOD1 mRNA may comprise MYOG, MYF5, MYF6, PAX3, PAX7, or any combination or variant thereof. An siRNA may comprise POU5F1 (OCT3/4), SOX2, NANOG, SEEA-4, KLF4, TRA-1-60, or any combination or variant thereof. The two or more different types of nucleic acid molecules comprise the MYOD1 mRNA as described anywhere herein and may comprise cDNA and/or siRNA. A cDNA may comprise MYOG, MYF5, MYF6, PAX3, PAX7, or any combination or variant thereof. The two or more different nucleic acids comprise the MYOD1 mRNA as described anywhere herein and may comprise a non-MYOD1 mRNA, cDNA, miRNA, tRNA, siRNA, uRNA, saRNA, or any variant, combinations, or analogs thereof.

One or more genes may be targeted and modulated with one, two, or a plurality of nucleic acid molecules. One or more genes may comprise greater or equal to about 1, 2, 3, 4, 5, 6, 7, 8, 9, 10 genes, or more. Modulating expression of one or more genes in said stem cells or said somatic cells may comprise enhancing expression of a first gene of the at least two genes, and inhibiting expression of a second gene of the at least two genes.

### 4. Biomaterial

Many cultured meat technologies may focus on satellite cell culture with cells grown in two-dimensional flasks or microcarriers in suspension. As provided herein, three-dimensional (3D) scaffolding and tissue engineering platforms may be used to facilitate large-scale growth. A food-safe scaffold may provide structural support and guide the growth of the cultured cells into the desired structure and/or texture analogous with the equivalent food product produced using conventional methods. Cell or tissue culture may comprise growing the population of cells on scaffolds within a bioreactor.

In some aspects, the present disclosure provides a method of generating an edible meat product from stem cells or somatic cells. The method may comprise bringing stem cells or somatic cells in contact with a degradable scaffold; culturing the cells to generate cultured cells; subjecting at least a subset of the cultured cells to one or more expansion processes to generate expanded cells; subjecting at least a subset of the expanded cells to a differentiation process in the presence of the degradable scaffold and with the use of the non-human animal MYOD1 mRNA as described anywhere herein, and optionally a growth factor, to generate a tissue; and generating an edible meat product using the tissue, which edible meat product may optionally comprise at least a portion of the degradable scaffold. A scaffold may be engineered to enhance stem cell or somatic cell proliferation, direct cell differentiation into a relevant lineage, or modulate flavour, texture, and tensile elasticity of the final meat product. In some embodiments, the nucleic acid comprises the non-human animal MYOD1 mRNA as described anywhere herein.

### 5. Bioreactor

Cells may be cultured and expanded to a desired quantity such as in a scalable manner using bioreactors to enable large-scale production. A bioreactor apparatus may provide a scalable method for differentiating and expanding stem cells or somatic cells into tissue and with the requisite growth needed for industrial production. Further, the mechanical conditioning of such an apparatus may provide a uniform method of producing a bio-artificial muscle that simulates standard meat in terms of its appearance, texture, and flavour at a competitive price. For example, some methods of producing cultured meat for human consumption comprise: a) obtaining a population of self-renewing cells derived from an animal; b) culturing the population of self-renewing cells in culture media comprising scaffolds within a bioreactor; c) inducing differentiation using factors comprising the MYOD1 mRNA as described anywhere herein in the population of cells to form at least one of terminally differentiated cells comprising myocytes and optionally adipocytes within a bioreactor; and d) culturing the cells into tissue within a bioreactor thus processing the population of cells into meat for human consumption.

A bioreactor system may comprise at least one bioreactor, bioreactor tank, or reactor chamber. Cell culturing, differentiation and/or expansion may each be conducted in a separate bioreactor chamber. In some examples, all processes (e.g., culturing, expansion, differentiation) may be performed in the same bioreactor chamber. A bioreactor system may be suitable for large-scale production of cultured cells for generation of food products. Cells may be cultured on a batch basis. Alternatively, or in combination, cells may be cultured continuously. In both batch and continuous cultures, fresh nutrients may be supplied to ensure the appropriate nutrient concentrations for producing the desired food product. A bioreactor system may produce at least a certain quantity of cells per batch. A bioreactor system may produce a batch of about 1 billion cells to about 100,000,000 billion cells. A bioreactor system may produce a batch of cultured cells during a certain time period. For example, in some cases, a bioreactor system may produce a batch of cultured cells at least once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, or more. A bioreactor system may produce a batch of cultured cells having at least a certain mass. Sometimes, the mass is measured as dry weight with excess media or supernatant removed. A bioreactor system may produce a batch of cultured cells of about 1 kilogram (kg) to about 100,000 kg. In certain instances, a bioreactor system produces a batch of at least about 1 kg. Differentiation may occur in the final bioreactor or may occur in a previous bioreactor. Differentiation of a stem cell or progenitor cell or transdifferentiation of a somatic cell into a terminally differentiated cell may take approximately 14-21 days or more. For example, a stem cell may be differentiated into a tissue comprised of skeletal muscle myocytes after 17 days of appropriate culture in a bioreactor. When a tissue has matured, it may be extracted as the food product. A mature tissue may comprise mature skeletal muscle fibers as part of the final meat product.

Expansion and differentiation phases may use one or different types of media. Media and growth conditions may be optimized using different media, temperatures, conditions, or compositions. One or multiple media storage tanks may be used to store one or multiple types of media

In some aspects, the present disclosure provides a method for producing an edible meat product. The method may comprise modulating expression of one or more genes in stem cells or somatic cells in a transient and non-integrative manner using one or more or two or more different compositions (e.g., ectopic differentiation factors) to generate progenitor cells, culturing at least a subset of the progenitor cells to generate cultured cells, and differentiating at least a subset of the cultured cells to generate terminally differentiated cells using the MYOD1 mRNA as described anywhere herein, to produce the edible meat product. The culturing and differentiating may be performed in the same bioreactor chamber or may be performed in different bioreactor chambers. A terminally differentiated cell comprises muscle cells, and optionally fat cells, bone cells, endothelial cells, smooth muscle cells, neural cells, somite cells, or a combination thereof.

Ectopic differentiation factors may induce differentiation in a transient and non-integrative manner using non-native induction through biochemical systems. Ectopic differentiation factors may comprise nucleic acids, polypeptides, small molecules, growth factors, or any combination thereof. A cultured stem cell or progenitor cell or somatic cell may be differentiated by arresting the cell cycle of the cell. Ectopic differentiation factors may arrest the cell cycle of cells by reducing or removing growth factors. Ectopic differentiation factors may arrest the cell cycle of cells through reducing or removing growth factors from a subset of cultured cells. Growth factors may be reduced or removed from a subset of cultured cells. Self-renewal and pluripotency of stem cells may be governed by extrinsic signals mediated by an endogenous pluripotency gene regulatory network consisting of a set of core transcription factors such as Oct3/4 or Sox2. Transcription factor interactions may regulate genomic functions by establishing both negative and positive feedback loops and transcription by recruiting activators and repressors to modulate the transcriptional machinery. Maintaining stem cell characteristics of self-renewal and differentiation in pluripotent stem cells may require distinct extrinsic signaling pathways including leukemia inhibitory factor (LIF), FGF/extracellular signal-regulated kinase (ERK) pathway, Wnt/glycogen synthase kinase 3 (GSK3), and transforming growth factor-beta (TGF-β) signaling. Growth factors which may influence the differentiation of stem or progenitor cells may comprise LIF, FGF, BMP, activin, MAPK, TGF-β, and NRG-1. Leukemia Inhibitory Factor may be a polyfunctional glycoprotein with actions on a broad range of tissue and cell types, including induction of differentiation in a number of myeloid leukemic cell lines, suppression of differentiation in normal embryonic stem cells, stimulation of proliferation of osteoblasts and haemopoietic cells. LIF may be necessary in establishing iPSCs from differentiated somatic cells. The addition of LIF to cell culture may improve the reprogramming of iPSCs from somatic cells as well as aid in the maintenance of stem cell proliferation. Activated fibroblast growth factor (FGF) signaling may sustain stem cells capabilities by promoting self-renewing proliferation and inhibiting cellular senescence. The removal of LIF may lead to the reversible conversion of embryonic stem cells from a naïve state to four FGF receptors/ERK-committed early differentiation states with features characteristic of primed pluripotency. Bone morphogenetic proteins (BMPs) through the SMAD-inhibitor of differentiation pathway with LIF may retain stem cell self-renewal and differentiation potential in stem cells. Inhibition of MAPK/ERK signaling pathway activation downstream of FGF signaling may improve stem cell stability and stemness. The FGF4/ERK signaling pathway activation may be necessary in multi-lineage differentiation of stem cells. FGF2 and Activin may enhance the expression of Oct4, thereby allowing the reversion from primed to naïve state of pluripotency in stem cells. TGFβ/activin/nodal signals via SMAD2/3 may be associated with stem cell pluripotency and may be required for the maintenance of primed stem cells and progenitor cells. Arresting the cell cycle of stem or progenitor cells may occur by reducing or removing serum levels in a solution in which the culturing is conducted. For example, replacing media comprising serum molecules with serum-free media may arrest the cell cycle of an iPSC and enhance the differentiation potential of the cell.

Culture conditions in a bioreactor may comprise static, stirred, or dynamic flow conditions. A bioreactor may be scaled in size to produce greater volume of cells or to allow greater control over the flow of nutrients, gases, metabolites, and regulatory molecules. A bioreactor may provide physical and mechanical signals such as compression, stretch, or alterations in flow to stimulate cells to produce specific biomolecules or to differentiate into a specific cell type. Unlike tissues derived from whole animals, tissues grown ex vivo or in vitro may have never been exercised (e.g. never been used to move a leg) and thus may have differences in flavour or texture without stimulation which may mimic the effects of exercise. A cell or tissue culture, or whole meat product may be exposed to a stimulus to increase the similarity in texture or flavour between meat grown ex vivo or meat derived from a whole animal. A cell or tissue culture may be exposed to a mechanical or electrical stimulus.

A meat product may comprise a meat having a certain ratio of fast twitch and slow twitch muscle cells and/or fibers. A meat product may comprise myocytes or skeletal muscle cells having a certain ratio or proportion of fast twitch (type II) and slow twitch (type I) muscle fibers. Slow twitch muscle fibers may exhibit low-intensity contractions fueled by the oxidative pathway and demonstrate relatively higher endurance, while fast twitch muscle fibers may have higher intensity contractions fueled by the glycolytic pathway. Fast twitch muscles may be characterized by high glycolytic and anaerobic muscle fibers. The ratio of fast twitch and slow twitch muscle fibers in muscle tissue may play a role in the taste, color, texture, and other culinary properties of the meat.

The bioreactor system may enable the culturing of cells for food production in a pathogen-free environment. Cells may be grown in a culture environment free of dangerous contaminants that affect human health. Cell culture plates, flasks, and bioreactors may provide cell culture conditions free of dangerous pathogens (e.g. H1N1), parasites, heavy metals, or toxins (e.g. bacterial endotoxins, pesticides, etc.). A cell culture system may not utilize antibiotics, in contrast to traditional livestock agriculture. A differentiation factor, media component or nucleotide molecule, or otherwise induction modality used in cell culture may be transient or may be removed before the cells or tissues are processed into a food product.

An edible meat product may be in a unit form of approximately or greater than 50 grams (g). An edible meat product may be in a unit form of at least about 1 g, 2 g, 3 g, 4 g, 5 g, 6 g, 7 g, 8 g, 9 g, 10 g, 15 g, 20 g, 25 g, 30 g, 35 g, 40 g, 45 g, 50 g, 60 g, 70 g, 80 g, 90 g, 100 g, 150 g, 200 g, 250 g, 300 g, 350 g, 400 g, 450 g, 500 g, 600 g, 700 g, 800 g, 900 g, 1000 g, or more than 1000 g. An edible meat product may be in a unit form of less than 1 g. A hamburger patty for example, may have a precooked weight of 85 g-113 g (3-4 ounces) if served diner style or 198 g-226 g (7-8 ounces) if served in a heavier pub-style.

### EXAMPLES

### Overview of Cell Culture Methodology in Producing an Edible Meat Product

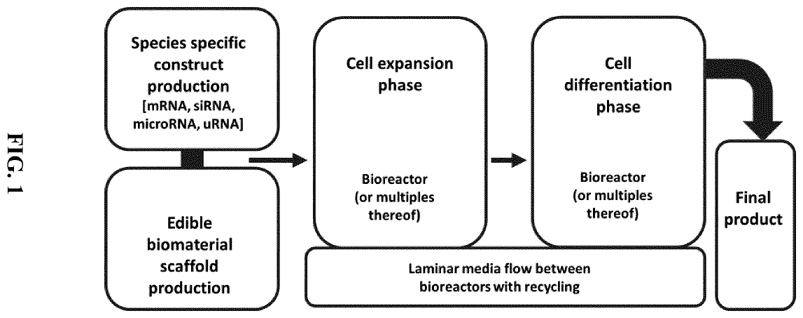

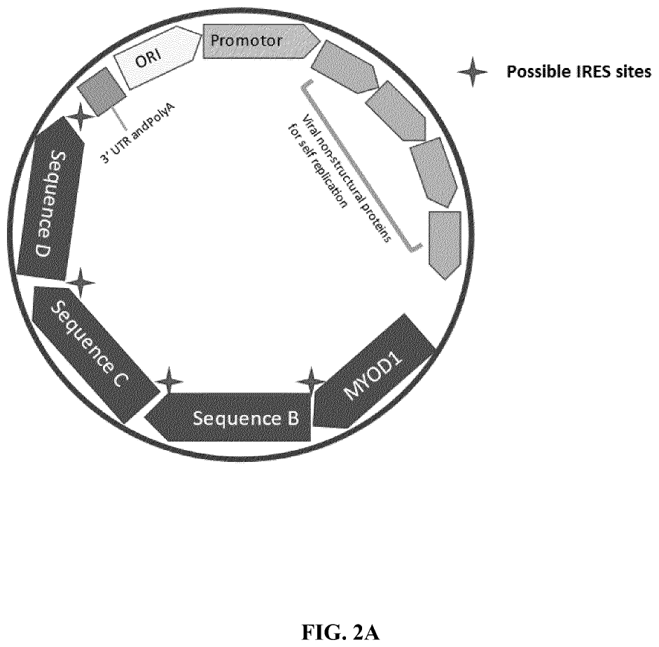

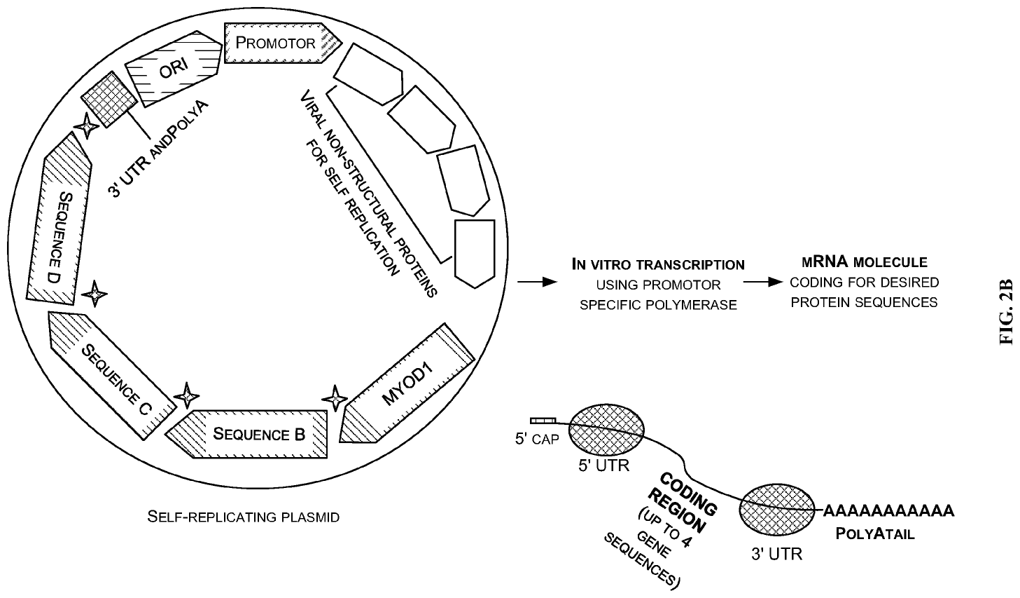

As illustrated in FIG. 1, an edible biomaterial scaffold is produced either separately to or in parallel to developing species-specific construct production for mRNA, siRNA, miRNA, saRNA or uRNAs. Cells are seeded on the edible scaffold which is held in the differentiation bioreactor. Cells are expanded in one or multiple bioreactors. These reactors are either a single vessel bioreactor or may comprise a plurality of bioreactor vessels. An expansion bioreactor is in fluid contact with laminar media flow and media recycling with either a single vessel bioreactor or plurality of bioreactor vessels for cell differentiation. Cells may remain or may be transferred to one or multiple bioreactors for differentiation. Cell differentiation may comprise an alteration of media, genetic manipulation, or ectopic differentiation factors being added during culture. Differentiated cells are then grown and matured into complex tissue with dense extracellular matrix on the scaffolds, at which point the tissue may be removed from the reactor. The resulting tissue may be used directly as an edible meat product or may be processed further into a meat product.

### Stem Cell Expansion and Differentiation Methods

Porcine iPSCs are maintained and expanded in iPSC medium (KO DMEM supplemented with 10% KO serum replacement, 10 ng/mL bFGF2, and/or 10 ng/ml human LIF, 0.1 mM non-essential amino acids, 2 mM glutamine) either on tissue culture treated plates (adherent cultures) or in non-tissue culture dishes on an orbital platform (suspension cultures). Cells are seeded onto tissue culture treated plates and/or coverslips (for analysis), and differentiation commences at 60% confluency.

Briefly, the necessary components are mixed with reduced serum-replacement medium and combined with the transfection reagent of choice for 10 minutes at room temperature, before being added to the cells. Cells are incubated at 37° C. for 24 hours, and the process repeats for 3 consecutive days. The components include but are not limited to mRNA, siRNA, cDNA, saRNA and/or miRNA. GFP/RFP/YFP tagged constructs or scrambled controls are used as transfection controls. In the present invention, components comprising mRNA of SEQ ID NO: 1 are mixed with culturemedium and combined with the transfection reagent of choice for 10 minutes at room temperature, before being added to the cells.

Transfection is carried out using either of the following technologies: traditional chemical based methods (e.g. Lipofection), non-chemical methods (e.g. electroporation or nucleofection), nanoparticle methods (e.g. liposome, polymer nanoparticles, micelles, or lipid-nanoparticles), or by magnet assisted transfection. The diverse nature of nucleotides affects the delivery method chosen as can be seen in the difference of nucleotide lengths, double vs single stranded nucleic acids, and the dose range of nucleotides: Silencing RNA (siRNA): 20-40 bps, double stranded RNA (dsRNA) molecule, messenger RNA (mRNA): range of 500 bp-2-4 kbp, single stranded RNA (ssRNA) molecule. Dose range of nucleotides: 0.5 μg/mL-50 μg/mL per nucleotide (DNA, RNA, mRNA, siRNA, saRNA). For example, mRNA and siRNA may be encapsulated together using a nanoparticle transfection option.

Cessation of transfection simultaneous with a reduced serum-replacement media directs cells down a myogenic lineage, with maturation of cultures over a course of 14-50 days promoting the formation of multinucleated myotubes.

Transfections are carried out in 2D (with or without biomaterial) or 3D (including but not limited to: spheroid, embryoid bodies, suspension or adherent, with or without biomaterial) culture conditions. Maturation of cultures are carried out in the described 2D or 3D conditions, with or without biomaterial, or with or without electrical stimulation or contractile tension forces to promote maturation of myogenic fibers.

On the 4th day, cells are switched to myogenic medium (KO DMEM supplemented with 10% serum replacement, 1 mM non-essential amino acids, 2 mM glutamine, 0.1 mM β-mercaptoethanol), to promote maturation and expansion. Cells are taken for analysis between 7- and 21-days post treatment.

Analysis may be conducted using immunohistological staining. Cells on coverslips may be fixed with 4% paraformaldehyde and treated to permeabilize using Triton-x 4% overnight at 4° C. Primary antibodies are all added directly to the blocking serum overnight at room temperature. Secondary antibodies are subsequently added in PBS for 2-4 hours at room temperature. For muscle specific staining, the primary antibody targets used include: myosin heavy chain/MYH3, MYOD1, PAX7, desmin, MYOG. Analysis is conducted using a Leica LAS X Widefield System and Leica Application Suite X (LAS X) or EVOS M5000.

Gene expression by PCR can also be conducted according to standard protocols using the below primer sequences as examples

PRIMERS

| | | | |
|---|---|---|---|
| MYOD1-F1 | AGCACTACAGTGGCGACTCA | 25 nm | STD |
| MYOD1-R1 | GCTCCACTATGCTGGACAGG | 25 nm | STD |
| MYOD1-F2 | CCTACTGTGGGCCTGCAAG | 25 nm | STD |
| MYOD1-R2 | GGATCTCCACCTTGGGCAAC | 25 nm | STD |
| PAX7-F | CCGTGTTTCCCATGGTTGTG | 25 nm | STD |
| PAX7-R | GAGCACTCGGCTAATCGAAC | 25 nm | STD |
| GAPDH-F | ATCACTGCCACCCAGAAGACT | 25 nm | STD |
| GAPDH-R | CATGCCAGTGAGCTTCCCGTT | 25 nm | STD |
| MYOGENIN-F | CTACAGGCCTTGCTCAGCTC | 25 nm | STD |
| MYOGENIN-R | AGATTGTGGGCGTCTGTAGG | 25 nm | STD |

PCR CYCLING

| PCR Step | Temperature ° C. | Duration |
|---|---|---|
| Denature template | 94° C. | 1 min |
| Anneal primers | 55° C. | 2 min |
| Extension | 72° C. | 3 min |

Other mRNAs/miRNAs/siRNAs/saRNAs may be used in permutations to this methodology. Experimental changes may use the same materials and methods, but different compounds may be introduced.

### Transient Expression of MYOD1 in Porcine iPSCs

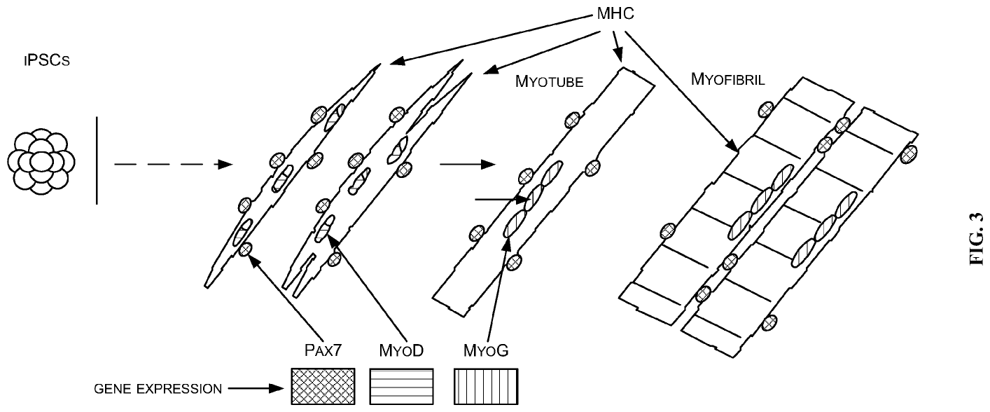

Human MYOD1 is transiently expressed in porcine iPSCs for 3 consecutive days. Cells are matured for a further 7 days at which point we expect at least 60% of cells to have maintained the MYOD1 expression, and should be immunoreactive for either MYOD1 or MyHC (myosin heavy chain). MYOD1 forced expression in porcine cells, results in the differentiation of iPSCs to skeletal muscle myocytes. Some cells may retain SOX2 expression signifying early differentiation stages with a remaining window of pluripotency. Over time it is expected that maturation of the muscle progenitor cells will result in an increase in myogenic markers and a decrease or loss of progenitor stem cell markers. Relative gene expressions during development are shown in the schematic of FIG. 3. This may be observed in all developmental stages of differentiation by PCR or immunohistochemistry. Positive and negative controls are used to standardise all experiments, and may include sample animal tissue, and an established differentiation control e.g. small molecules to differentiate porcine iPSCs to skeletal muscle myocytes, for which there is a working model with 60-70% efficiency rate.

In line with this, experiments have been carried out as follows:

### i) Gene Expression Analysis of Porcine Muscle Differentiation

Method: mRNA polyplex was prepared following manufacturer's instructions. Briefly, standard lipid-based transfection reagents were mixed with MYOD1-mRNA of SEQ ID NO: 1 for 20 minutes at room temperature, before being added to the cells in culture. Media was changed following 6 hours, and cells were maintained in culture for 7 days. On day 7, the samples were lysed in trizol and qPCR analysis was conducted using SYBRgreen.

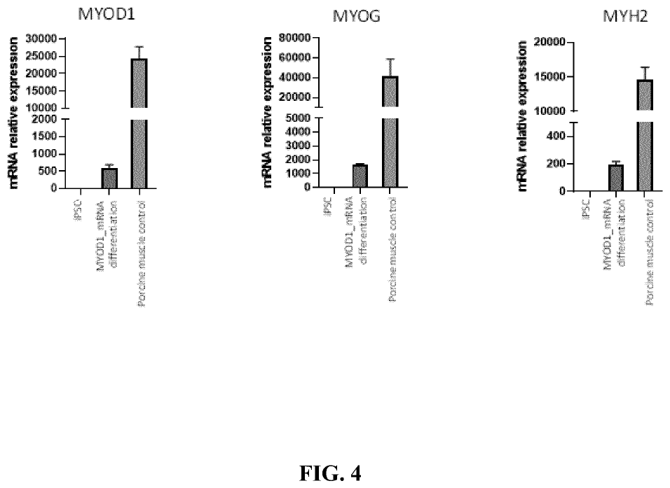

Results: Results are shown in FIG. 4. Using standard lipid based approaches, MYOD1-mRNA of SEQ ID NO: 1 was delivered to porcine iPSCs and analysed by qPCR after 7 days. Gene expression of MYOD1 and downstream gene expression of MYOG and MYH2 are elevated, showing initiation of the muscle regulatory cascade. Gene expression is relative to the stage of differentiation, with MYOD1 expression (the ‘inducer’) peaking at the point of transfection and then remaining in lower levels between days 7-14. MYOG expression starts to rise soon after the cascade is initiated, and MYHC2 rises as the muscle cells mature and become more established. iPSC are negative control, porcine muscle tissue positive control. For reference of gene expression at certain developmental stages, please refer to FIG. 3.

### ii) Protein Analysis of Porcine Muscle Differentiation

Method: mRNA polyplex was prepared following manufacturer's instructions. Briefly, standard lipid-based transfection reagents were mixed with MYOD1-mRNA of SEQ ID NO: 1 for 20 minutes at room temperature, before being added to the cells in culture. Media was changed following 6 hours, and cells were maintained in culture for 7 days. Samples were dissociated and frozen at each time course. Samples were prepared for western blotting procedures according to manufacturer's instruction. Briefly, samples were prepared in sample buffer and BCA assay performed. 14 ug protein was loaded per sample and the gel was run at 240V for 20 minutes. Samples were transferred to membrane and subsequently blocked and probed using blocking buffer and antibodies. Positive loading control used was tubulin.

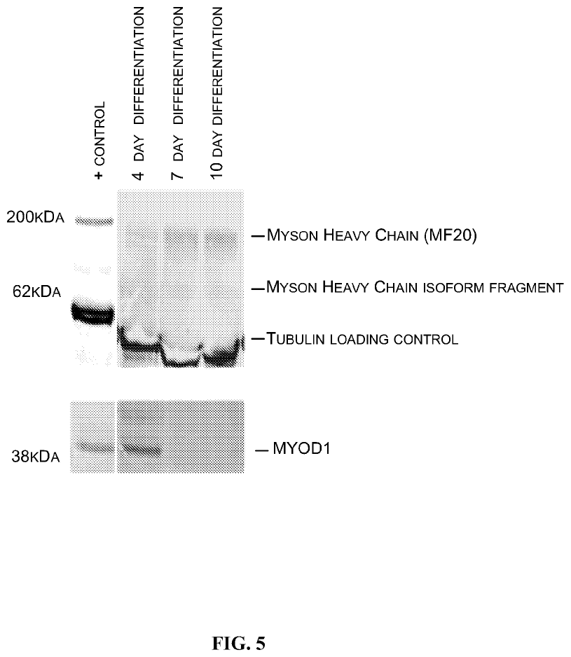

Results: Results are shown in FIG. 5. Cells were taken for western blot analysis after 4, 7 and 10 days in culture. Results show that as the cells mature in culture, the protein expression of myosin heavy chain increases. Downstream protein expression is paramount to muscle cell development following introduction of MYOD1-mRNA, with myosin heavy chain being one of the key proteins to show successful differentiation over a 10 day time-course. MYOD1 expression appears to fluctuate and decreases over time, but Myosin heavy chain increases. Porcine muscle was used as a positive control. Refer to FIG. 1) for gene and protein development for reference.

### iii) Immunohistochemical Analysis of Porcine Muscle Cells

Methods: mRNA polyplex was prepared following manufacturer's instructions. Briefly, standard lipid-based transfection reagents were mixed with MYOD1-mRNA of SEQ ID NO: 1 for 20 minutes at room temperature, before being added to the cells in culture. Media was changed following 6 hours, and cells were maintained in culture for 14 days. Cell were fixed and stained for myosin heavy chain, MYOD1 and DAPI.

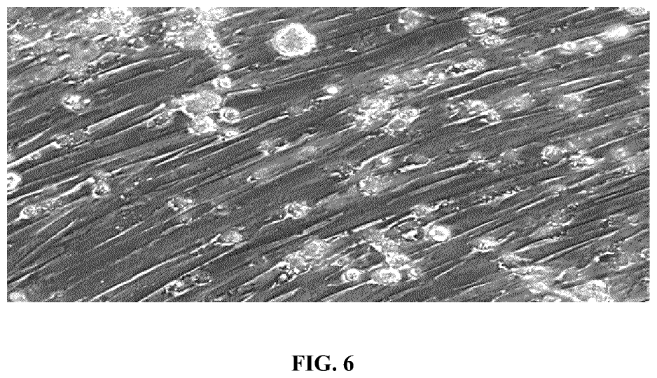

Results: Results are shown in FIG. 6. Skeletal muscle cells are strongly immunoreactive for myosin heavy chain (grey scale), MYOD1 (not shown) and DAPI (demonstrating multinucleation). The staining highlights the strong elongated muscle morphology profile for myocytes.

### iv) Microscopic Analysis of Porcine Muscle Cells

Methods: mRNA polyplex was prepared following manufacturer's instructions. Briefly, standard lipid-based transfection reagents were mixed with MYOD1-mRNA of SEQ ID NO: 1 for 20 minutes at room temperature, before being added to the cells in culture. Media was changed following 6 hours, and cells were maintained in culture for 14 days. Cell were fixed and examined by confocal microscopy.

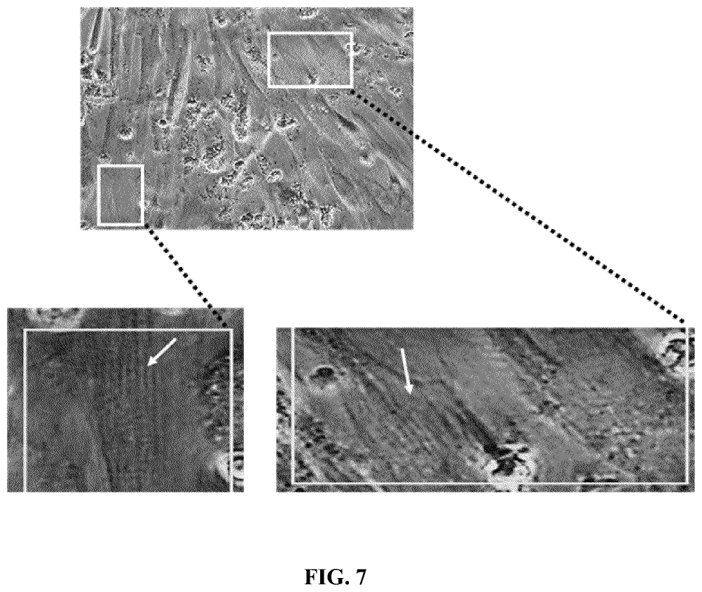

Results: Results are shown in FIG. 7. The myogenic cells show striation as early as 14 days in culture following transfection with MYOD1_mRNA. The visible striations within the elongated myocytes (FIG. 6) show maturation of the fibres, which promotes formation of myotubes and demonstrates communication between cells in culture.

### Cell Culture Using a Scalable Bioreactor

A bioreactor system is designed such that there are two or more bioreactors in which iPSC expansion occurs, and multiple bioreactors in which iPSC differentiation occurs (FIG. 1).

Cells are first grown within the first bioreactor(s) of a size ‘x’ for a period of time suitable to cell doubling time. This approximate time value comes from experience culturing these cells in both suspension and adherent culture. The cells are passaged to a further bioreactor(s) of increased volume according to cell doubling time. The iPSCs are further expanded within these bioreactors. The cells are then further passaged into further bioreactors which may contain a biomaterial scaffold. It is in these final bioreactors that differentiation is initiated and or maintained. The approximate time to differentiate these cells to produce mature skeletal muscle fibers is approximately 14-21 days in the absence or presence of biomaterial scaffolds within the bioreactor. Once the mature skeletal muscle fibers have been produced, they are removed from the system and the meat is extracted. This part of the design in particular is subject to change and enhancement.

Since expansion and differentiation phases call for different media component mixes, 2 media storage tanks are required. Media within these tanks will be stored at 4° C. and the component to induce differentiation will be added into the appropriate media storage tank when required. Storage at either 4° C. or −20° C. prevents component degradation. Some differentiation factors and small molecules degrade rapidly if temperatures exceed −20 degrees.

### Media Recycling and Perfusion

After the initial passaging of cells into each bioreactor, media recycling will be employed. The media is under continuous laminar flow within and between bioreactor vessels. The bioreactor agitation will ensure consistent mixing and perfusion of the media within the vessel proper. Spent media will be removed via a designed exit port and subjected to dialysis with the necessary membranes for filtering specific metabolites and components. The media is then replenished with the necessary components and fed back into the bioreactor vessel. Gas and metabolite exchange will be tightly controlled using dialysis and membranes.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A non-human MYOD1 messenger RNA (mRNA), wherein the MYOD1 mRNA comprises a nucleic acid sequence having 70% or more sequence identity to the nucleic acid sequence of SEQ ID NO: 1.

2. The mRNA of claim 1, wherein the mRNA is a porcine MYOD1 mRNA.

3-4. (canceled)

5. The mRNA of claim 1, wherein the mRNA comprises at least one nucleic acid modification in the 5′UTR, 3′UTR, or length of the polyA-tail.

6. A composition comprising the mRNA of claim 1 and at least a second RNA molecule.

7. The composition of claim 6, further comprising an siRNA molecule.

8. The composition of claim 7, wherein the siRNA molecule comprises POU5F1 (OCT3/4), SOX2, NANOG, SEEA-4, KLF4, or TRA-1-60.

9. The composition of claim 6, further comprising an non-MYOD1 mRNA.

10. The composition of claim 9, wherein the non-MYOD1 mRNA comprises MRF4, PAX7, PAX3, MYOG, MYF5, or MYF6 mRNA.

11. (canceled)

12. A plasmid comprising the mRNA of claim 1.

13. The plasmid of claim 12, further comprising an non-MYOD1 mRNA.

14. The plasmid of claim 13, wherein the non-MYDO1 mRNA comprises MRF4, PAX7, PAX3, MYOG, MYF5, or MYF6 mRNA.

15. Use of the mRNA of claim 1 in the production of an edible meat product.

16. A method for differentiating stem cells or transdifferentiating somatic cells, to produce an edible meat product, the method comprising:
(a) delivering nucleic acid molecules comprising two or more different ribonucleic acid (RNA) molecules into said cells, wherein the RNA molecules comprise non-human MYOD1 messenger RNA (mRNA) comprising or a nucleic acid sequence having 70% or more sequence identity to the nucleic acid sequence of SEQ ID NO: 1;
(b) modulating expression of one or more genes in said cells with aid of said nucleic acid molecules to cause at least a subset of said cells to yield one or more progenitor cells following delivery of said nucleic acid molecules, wherein upon said modulating, said nucleic acid molecules are not integrated into a genome of said cells;
(c) culturing said yielded one or more progenitor cells to generate one or more cultured cells; and
(d) differentiating said generated one or more cultured cells to generate one or more terminally differentiated cells to produce said edible meat product.

17. The method of claim 16, wherein the MYOD1 mRNA is porcine MYOD1 mRNA.

18-19. (canceled)

20. The method of claim 16, wherein the MYOD1 mRNA comprises at least one nucleic acid modification in the 5′UTR, 3′UTR, or polyA-tail.

21. The method of claim 16, wherein (d) further comprises producing a tissue from said one or more terminally differentiated cells before producing said edible meat product.

22. The method of claim 21, wherein said tissue comprises muscle tissue, fat tissue, neural tissue, vascular tissue, epithelial tissue, connective tissue, bone tissue, or a combination thereof.

23. (canceled)

24. The method of claim 21, wherein (d) further comprising co-culturing said one or more terminally differentiated cells to generate a three-dimensional tissue before producing said edible meat product.

25. The method of claim 23, wherein said at least two types of terminally differentiated cells comprise muscle cells, fat cells, somite cells, neural cells, endothelial cells, smooth muscle cells, bone cells, or a combination thereof.

26. The method of any one of claims 16 to 25, wherein said RNA molecules comprise MYOG, MYF5, MYF6, PAX3, or PAX7, non-MYOD1 messenger RNA (mRNA), microRNA (miRNA), transfer RNA (tRNA), silencing RNA (siRNA), self-amplifying RNA (saRNA), any combinations, or chemically modifications, or variants thereof.

27-67. (canceled)


SEQUENCE LISTING (8 sequences)

SEQ ID NO: 1 - DNA, 960 bp, Artificial Sequence
  synthetic construct
atggagctgctgagcccccccctgagggacgtggacctgaccggccccgacggcagcctgtgcaacttcgccaccgccgacgacttctacgacgacccctgcttcgacagccccgacctgaggttcttcgaggacctggaccccaggctggtgcacgtgggcgccctgctgaagcccgaggagcacagccacttccccgccgccgcccaccccgcccccggcgccagggaggacgagcacgtgagggcccccagcggccaccaccaggccggcaggtgcctgctgtgggcctgcaaggcctgcaagaggaagaccaccaacgccgacaggaggaaggccgccaccatgagggagaggaggaggctgagcaaggtgaacgaggccttcgagaccctgaagaggtgcaccagcagcaaccccaaccagaggctgcccaaggtggagatcctgaggaacgccatcaggtacatcgagggcctgcaggccctgctgagggaccaggacgccgccccccccggcgccgccgccgccttctacgcccccggccccctgccccccggcaggggcggcgagcactacagcggcgacagcgacgccagcagccccaggagcaactgcagcgacggcatgatggactacagcggcccccccagcggcgccaggaggaggaactgctacgacggcacctactacagcgaggcccccagcgagcccaggcccggcaagaacgccgccgtgagcagcctggactgcctgagcagcatcgtggagagcatcagcaccgagagccccgccgcccccgccctgctgctggccgacacccccagggagagcagccccggcccccaggaggccgccgccggcagcgaggtggagaggggcacccccacccccagccccgacgccgccccccagtgccccgccagcgccaaccccaaccccatctaccaggtgctgtga

SEQ ID NO: 2 - DNA, 675 bp, S. domesticus
atggagctgtatgagacatccccctacttctaccaggaaccccacttctatgacggggaaaactacctgcccgtccacctccagggctttgagccaccaggctacgagcggactgagctgagtctgagccctgaggcccgagtgcccctggaagataaggggctggggacccccgagcactgcccaggccagtgcctgccgtgggcatgtaaggtgtgtaagaggaagtccgtgtctgtggaccgtcggcgggccgccacgctgagggagaagcgcaggctcaagaaggtgaatgaggcctttgaggccctgaagaggagcaccctgctcaaccccaaccagcggctgcccaaggtggagatcctgcgcagcgccatccagtacatcgagtgcctgcaggccctgctcagctccctcaaccaggaggagcgagacctccgctaccgaggcgggggcgggccgcagccaggggtgcccagtgaatgcagttcccacagcgcctcctgcagtccagaatggggcagtgcactggagttcggccccaacccaggggatcatctgctcacagctgaccctacagatgcccacaatctgcactccctcacctccatcgtggacagcatcacagtggaggatgtggctgtggccttcccagatgaaaccatgcccaactga

SEQ ID NO: 3 - DNA, 579 bp, S. domesticus
atggacaacctgagggagaccttcctgagcctggaggacggcctgggcagcagcgacagccccggcctgctgagcagctgggactggaaggacagggccggccccttcgagctgaaccaggccagccccagccagagcctgagccccgcccccagcctggagagctacagcagcagcccctgccccgccgtggccggcctgccctgcgagcacggcggcgccagcagcggcggcagcgagggctgcagcgtgggcggcgccagcggcctggtggaggtggactacaacatgctggccttccagcccacccacctgcagggcggcggcggccccaaggcccagaagggcaccaaggtgaggatgagcgtgcagaggaggaggaaggccagcgagagggagaagctgaggatgaggaccctggccgacgccctgcacaccctgaggaactacctgccccccgtgtacagccagaggggccagcccctgaccaagatccagaccctgaagtacaccatcaagtacatcggcgagctgaccgacctgctgaacaggggcagggagcccagggcccagagcgcc

SEQ ID NO: 4 - DNA, 765 bp, S. domesticus
atggacgtgatggacggctgccagttcagccccagcgagtacttctacgacggcagctgcatccccagccccgagggcgagttcggcgacgagttcgtgcccagggtggccgccttcggcgcccacaaggccgagctgcagggcagcgacgaggacgagcacgtgagggcccccaccggccaccaccaggccggccactgcctgatgtgggcctgcaaggcctgcaagaggaagagcaccaccatggacaggaggaaggccgccaccatgagggagaggaggaggctgaagaaggtgaaccaggccttcgagaccctgaagaggtgcaccaccaccaaccccaaccagaggctgcccaaggtggagatcctgaggaacgccatcaggtacatcgagagcctgcaggagctgctgagggagcaggtggagaactactacagcctgcccggccagagctgcagcgagcccaccagccccaccagcaactgcagcgacggcatgcccgagtgcaacagccccgtgtggagcaggaagagcagcaccttcgacagcatctactgccccgacgtgagcaacgtgtacgccaccgacaagaacagcctgagcagcctggactgcctgagcaacatcgtggacaggatcaccagcagcgagcagcccggcctgcccctgcaggacctggccagcctgagccccgtggccagcaccgacagccagcccgccacccccggcgccagcagcagcaggctgatctaccacgtgctg

SEQ ID NO: 5 - DNA, 726 bp, S. domesticus
atgatgatggacctgttcgagaccggcagctacttcttctacctggacggcgagaacgtgaccctgcagcccctggaggtggccgagggcagccccctgtaccccggcagcgacggcaccctgagcccctgccaggaccagatgccccccgaggccggcagcgacagcagcggcgaggagcacgtgctggccccccccggcctgcagcccccccactgccccggccagtgcctgatctgggcctgcaagacctgcaagaggaagagcgcccccaccgacaggaggaaggccgccaccctgagggagaggaggaggctgaagaagatcaacgaggccttcgaggccctgaagaggaggaccgtggccaaccccaaccagaggctgcccaaggtggagatcctgaggagcgccatcagctacatcgagaggctgcaggacctgctgcacaggctggaccagcaggagaagatgcaggagctgggcgtggaccccttcagctacaggcccaagcaggagaacctggagggcgccgacttcctgaggacctgcagcagccagtggcccagcgtgagcgaccacagcaggggcctggtgatcaccgccaaggagggcggcgccagcatcgacagcagcgccagcagcagcctgaggtgcctgagcagcatcgtggacagcatcagcagcgaggagaggaagctgccctgcgtggaggaggtggtggagaag

SEQ ID NO: 6 - DNA, 672 bp, S. domesticus
atggagctgtacgagaccagcccctacttctaccaggagcccaggttctacgacggcgagaactacctgcccgtgcacctgcagggcttcgagccccccggctacgagaggaccgagctgaccctgagccccgaggcccccggccccctggaggacaagggcctgggcacccccgagcactgccccggccagtgcctgccctgggcctgcaaggtgtgcaagaggaagagcgtgagcgtggacaggaggagggccgccaccctgagggagaagaggaggctgaagaaggtgaacgaggccttcgaggccctgaagaggagcaccctgctgaaccccaaccagaggctgcccaaggtggagatcctgaggagcgccatccagtacatcgagaggctgcaggccctgctgagcagcctgaaccaggaggagagggacctgaggtacaggggcggcggcggcccccagcccggcgtgcccagcgagtgcagcagccacagcgccagctgcagccccgagtggggcagcgccctggagttcagcgccaaccccggcgaccacctgctgaccgccgaccccaccgacgcccacaacctgcacagcctgaccagcatcgtggacagcatcaccgtggaggacgtgagcgtggccttccccgacgagaccatgcccaac

SEQ ID NO: 7 - DNA, 1509 bp, S. domesticus
atggccgccctgcccggcaccgtgcccaggatgatgaggcccgcccccggccagaactaccccaggaccggcttccccctggaggtgagcacccccctgggccagggcagggtgaaccagctgggcggcgtgttcatcaacggcaggcccctgcccaaccacatcaggcacaagatcgtggagatggcccaccacggcatcaggccctgcgtgatcagcaggcagctgagggtgagccacggctgcgtgagcaagatcctgtgcaggtaccaggagaccggcagcatcaggcccggcgccatcggcggcagcaagcccaggcaggtggccacccccgacgtggagaagaagatcgaggagtacaagagggagaaccccggcatgttcagctgggagatcagggacaggctgctgaaggacggccactgcgacaggagcaccgtgcccagcgtgagcagcatcagcagggtgctgaggatcaagttcggcaagaaggaggaggacgacgaggccgacaagaaggaggacgacagcgagaagaaggccaagcacagcatcgacggcatcctgggcgacaagggcaacaggctggacgagggcagcgacgtggagagcgagcccgacctgcccctgaagaggaagcagaggaggagcaggaccaccttcaccgccgagcagctggaggagctggagaaggccttcgagaggacccactaccccgacatctacaccagggaggagctggcccagaggaccaagctgaccgaggccagggtgcaggtgtggttcagcaacaggagggccaggtggaggaagcaggccggcgccaaccagctggccgccttcaaccacctgctgcccggcggcttcccccccaccggcatgcccaccctgcccccctaccagctgcccgacagcacctaccccaccaccaccatcagccaggacggcggcagcaccgtgcacaggccccagcccctgccccccagcaccatgcaccagggcggcctggccgccgccgccgccgccgccgacaccagcagcgcctacggcgccaggcacagcttcagcagctacagcgacagcttcatgaaccccgccgcccccagcaaccacatgaaccccgtgagcaacggcctgagcccccaggtgatgagcatcctgagcaaccccagcgccgtgcccccccagccccaggccgacttcagcatcagccccctgcacggcggcctggacagcgccaccagcatcagcgccagctgcagccagagggccgacagcatcaagcccggcgacagcctgcccaccagccagagctactgcccccccacctacagcaccaccggctacagcgtggaccccgtggccggctaccagtacggccagtacggccagaccgccgtggactacctggccaagaacgtgagcctgagcacccagaggaggatgaagctgggcgagcacagcgccgtgctgggcctgctgcccgtggagaccggccaggcctac

SEQ ID NO: 8 - DNA, 1334 bp, S. domesticus
actaattaaatgccatctgggtggctcctctgggtttttgagcccatcacccagttcagaccgagtcagaggccaaggaggagaacatgatgatggacctttttgaaactggctcctatttcttctatttggacggggaaaatgttaccctgcagcccctagaagtggcagaaggctctcctttgtatccagggagtgatggtaccctgtccccctgccaggaccaaatgcccccggaagctgggagcgacagcagtggagaggaacatgtcctggcgcccccaggcctgcagcctccccactgccccggccaatgtctgatctgggcttgcaagacctgcaagagaaaatctgccccaaccgaccgcaggaaggcggccactctgcgcgagaggaggcggctgaagaaaatcaacgaggccttcgaggcactgaagcggcggactgtggccaaccccaaccaaaggctgcccaaggtggagatcctgcggagcgccatcaactacatcgagaggttgcaggacctgctgcaccggctggatcagcaggacaaaatgcaggagctaggcgtggaccccttcagctacagacccaagcaagagaatcttgagggtgcggatttcctgcgcacctgcagctcccagtggccaagtgtttcggatcattccagggggctcgtgatgactgccaaggaaggagggacaaacattgattcgtcagcctcgagcagccttcggtgcctttcttccatcgtggacagcatttcctcggaggaacacacgctcccctgcgtcgaggaagtgggggagaaataactcgcggccggagacggtctccacgcagcagcaaaagcccaccctcctcctcctcctcctcctccgcctaatcctgtagatgaggtcacgttacgtgaatatttaggaaccctgactcaggagctcacgaaagggaaggggacatcttcgcaaagaaacttctcggaagctgttgcgcacgctcggaggagaagcctcgcagccttgggcttttcttcggcgaactgcgagtggcttagatctacagcagccttggtttttgctgggtgggctctgtaacatatttacgtttcctatggtgatccttttgtgccctgtgcaaaagaagttcattcctgtctaaagcaaagtgggaacgtcgcaactgttagtgggattgaatgtatttttgtaaataatcttagtactttcatttttttatgtcaacctaagaaatatattttaaacgtggagtgacgtattgtatacatagcgtgcaaggatcctggtattgttatattaaaaagataagtttctata